(19) United States
(12) Patent Application Publication
(10) Pub. No.: US 2005/0261250 A1
(43) Pub. Date: Nov. 24, 2005

(54) Compositions and methods for inhibiting bone resorption

(75) Inventors: Anastasia G. Daifotis, Westfield, NJ (US); Andrew Denker, Hoboken, NJ (US); Craig Ikeda, Harleysville, PA (US); Bogdan K. Matuszewski, North Wales, PA (US); Sid Mazel, Basking Ridge, NJ (US); Arturo G. Porras, Lansdale, PA (US); Art Santora, Watchung, NJ (US); Randal Alan Seburg, Collegeville, PA (US); Limin Zhu, Collegeville, PA (US); John Yates, Libertyville, IL (US); John D. Kirsch, Waynesburg, PA (US)

Correspondence Address: COVINGTON & BURLING;ATTN: PATENT DOCKETING, 1201 PENNSYLVANIA AVENUE, N.W., WASHINGTON, DC, 20004-2401, US

(73) Assignee: Merck & Co., Inc.,

(21) Appl. No.: 10/848,503

(22) Filed: May 19, 2004

(51) Int. Cl. A61K 31/675; A61K 31/59; A61K 31/663

(52) U.S. Cl. 514/89; 514/102; 514/167

(57) ABSTRACT

Disclosed are compositions and methods for preventing, inhibiting, reducing and treating conditions and diseases associated with abnormal bone resorption in mammals, including for example osteoporosis. Embodiments of compositions of the invention comprise a pharmaceutically effective amount of alendronate and vitamin D₃ suitable for once-weekly dosing. Compositions and methods of the invention provide vitamin D nutrition during bisphosphonate treatment to facilitate normal bone formation and mineralization while minimizing the occurrence of or potential for the complications associated with vitamin D insufficiency, such as hypocalcaemia and osteomalacia. Also disclosed are methods for manufacturing compositions of the present invention, for measuring stability and degradation of those compositions, and for measuring blood plasma levels of vitamin D.

### BACKGROUND OF THE INVENTION

1. Field of the Invention

The present invention relates to compositions comprising a bisphosphonate compound and a vitamin D compound. The present invention also relates to methods of using such compositions for example to treat, reduce, inhibit or prevent abnormal bone resorption in mammals. The present invention further relates to methods of making bisphosphonate and vitamin D compositions.

2. Related Art

A variety of disorders in humans and other mammals involve or are associated with abnormal bone resorption. Among the most common of these disorders is osteoporosis, which is a systemic skeletal disease characterized by a low bone mass and microarchitectural deterioration of bone tissue, with a consequent increase in bone fragility and susceptibility to fracture. Osteoporosis is becoming a worldwide pandemic, with marked increases in its occurrence coinciding with the worldwide increase of longevity.

A principal cell type responsible for bone resorption is the multinucleated cell called the osteoclast. Bisphosphonates are well known as selective inhibitors of osteoclastic bone resorption. Bisphosphonates are believed to bind to hydroxyapatite in bone and to inhibit the bone resorptive activity of osteoclasts through their intracellular action. See, e.g., H. Fleisch, Bisphosphonates In Bone Disease, From The Laboratory To The Patient, 4th Edition, Academic Press (2000). It has also been reported that bisphosphonates bind to bone and then are released into the resorption lacuna during resorption. After this they are taken up by the osteoclast, and subsequently inhibit the enzyme farnesyl diphosphate synthase. This intracellular action in turn prevents the isoprenylation (farnesylation and geranylgeranylation) of GTPases, signaling proteins that attach to membrane vesicles. The family of geranylgeranylated small GTPases include those that direct the formation of the ruffled border—the organelle of active bone resorption. See A. A. Reszka and G. A. Rodan, “Bisphosphonate Mechanism of Action,” Curr Rheumatol Rep. 5(1):65-74 (February, 2003).

Bisphosphonates are understood to be useful in preventing bone loss associated with a number of conditions. For example, bisphosphonates are known to be useful in the prevention of bone loss and in the treatment of diseases such as, but not limited to, osteoporosis, osteopenia, metastatic bone disease, multiple myeloma, periodontal disease, tooth loss, hyperparathyroidism, rheumatoid arthritis, Paget's disease, osteonecrosis, osteoarthritis, periprosthetic bone loss or osteolysis, and hypercalcemia of malignancy. All of these conditions are characterized by bone loss, resulting from an imbalance between bone resorption—i.e., breakdown—and bone formation.

Alendronate sodium is one of the most potent bisphosphonates currently available, and does not impair bone mineralization at doses which maximally inhibit bone resorption. It has also been found that the increase in bone mineral density observed with the administration of alendronate is positively associated with a decrease in vertebral and non-vertebral (including the hip) fractures, a decrease in spinal deformity and a retention of height. This indicates that when administered for a substantial period of time, alendronate decreases bone turnover acting positively to produce a strengthened bone. Alendronate sodium is approved in more than 90 countries for the treatment of osteoporosis in postmenopausal women. Alendronate sodium is also approved for the treatment of osteoporosis in men, glucocorticoid-induced osteoporosis, and Paget's disease of bone. Evidence suggests that other bisphosphonates such as ibandronate, minodronate, pamidronate, risedronate, tiludronate and zoledronate, have many properties in common with alendronate, including high potency as inhibitors of osteoclastic bone resorption.

Despite their therapeutic benefits, bisphosphonates are poorly absorbed (on the order of about 1%) from the gastrointestinal tract. See, e.g. B. J. Gertz et al., Clinical Pharmacology of Alendronate Sodium, Osteoporosis Int., Suppl. 3: S13-16 (1993) and B. J. Gertz et al., Studies of the oral bioavailability of alendronate, Clinical Pharmacology & Therapeutics, vol. 58, number 3, pp. 288-298 (September 1995). It is understood that food, as well as many other substances that may be ingested concomitantly (including beverages such as mineral water, and even some excipients used to formulate dosing vehicles) can adversely affect bisphosphonate absorption. Intravenous administration has been used to ensure that the entire dose reaches the circulation. However, intravenous administration is costly and inconvenient, especially when the subject must be given an intravenous infusion lasting several hours on repeated occasions. Unlike oral administration, intravenous administration of bisphosphonates is associated with acute renal injury if administered too rapidly.

If, instead of intravenous administration, oral administration of the bisphosphonate is desired, higher doses may be administered to compensate for the low bioavailability from the gastrointestinal tract. To offset this low bioavailability, it is generally recommended that the subject take the bisphosphonate on an empty stomach and fast for at least 30 minutes afterwards. However, many subjects find such fasting on a daily basis to be inconvenient.

Bisphosphonate therapy has been associated with hypocalcaemia. During treatment with bisphosphonates, the early inhibition of bone resorption can induce a decrease in serum calcium, which occurs within hours, days or weeks of the start of treatment. The serum calcium decrease can persist for many weeks to months following the initiation of treatment and can be prominent in subjects having a deficiency in vitamin D. The hypocalcaemia response to bisphosphonate therapy can occasionally be severe enough to be symptomatic and warrant clinical intervention, particularly in patients with hypoparathyroidism (See, e.g., Vasikaran, S. D., Ed., Bisphosphonates: An Overview with Special Reference to Alendronate, Ann. Clin. Biochem. (2001) 38: 608-623). As there are no substantial body stores of calcium outside of bone, the calcium required for new bone formed after treatment with alendronate is initiated must be absorbed from the diet—either from food or calcium supplements. Vitamin D is required for normal calcium absorption. Thus, adequate vitamin D and calcium intake is desirable for subjects using bisphosphonates. Adequate vitamin D levels become even more important when calcium needs are elevated due to the net influx of calcium into bone that occurs as a result of bisphosphonate therapy during effective osteoporosis treatment. As a result, adequate vitamin D and calcium intake is desirable for subjects using bisphosphonates.

Vitamin D compounds comprise a group of fat soluble secosteriods that are found in very few foods naturally, and they are photosynthesized in the skin of vertebrates by the action of solar UV radiation. While vitamin D may come in several forms, the most physiologically relevant forms are vitamin D₃ (cholecalciferol) and vitamin D₂ (ergocalciferol). The latter is formed when the yeast and plant sterol, ergosterol, is exposed to UV radiation, while the former originates from 7-dehydrocholesterol and is synthesized in the skin. The metabolic pathway for vitamin D₃ and vitamin D₂ is similar, and their biological efficacy in humans is similar, their main function being the maintenance of serum calcium and phosphorous concentrations within normal ranges. Vitamin D₃ is the obligate precursor of the hormone calcitriol (also called 1,25-dihydroxycholecalciferol or 1,25-dihydroxyvitamin D₃), whose principal action is to enhance the ability of the small intestine to absorb calcium, and retain phosphate from the diet. The hormone-like metabolite of ergocalciferol is 1,25-dihydroxyergocalciferol (1,25-dihydroxyvitamin D₂). When dietary calcium intake is insufficient to satisfy the body's needs, parathyroid hormone (PTH), along with the hormonal metabolite of vitamin D₃, calcitriol, mobilizes monocytic stem cells in the bone marrow to become mature osteoclasts. These osteoclasts are themselves stimulated by a variety of cytokines and other factors to increase the mobilization of calcium stores from the bone.

The naturally-occurring forms of cholecalciferol and ergocalciferol are biologically inactive precursors of the hydroxylated biologically active metabolites of vitamin D. Because vitamin D is lipid soluble, it may be stored in fat tissues in the body or metabolized to the principal storage metabolite 25-hydroxyvitamin D and stored in other organs. The 25-hydroxyvitamin D is transported in blood plasma and metabolized by the body when needed. Specifically, as shown in the example of cholecalciferol in FIG. 1, 7-dehydrocholesterol in the skin converts to pre-vitamin D₃ (an isomer of vitamin D₃, not shown in FIG. 1) upon exposure to sunlight, and then to vitamin D₃ (cholecalciferol). Cholecalciferol is then metabolized in the liver to form 25-hydroxycholecalciferol, also known as 25-hydroxy vitamin D₃, and this is further metabolized in the kidney to the hormonal form 1,25-dihydroxycholecalciferol, also known as calcitriol. Although not depicted in FIG. 1, other metabolites of vitamin D (D₂ or D₃) include 1α-hydroxy vitamin D, 24,25 dihydroxy vitamin D, and 1α,24,25-trihydroxy vitamin D. Only calcitriol is fully biologically active; cholecalciferol and the metabolites identified above show little or no biological activity.

A primary biological function of vitamin D (both D₂ and D₃) is to help maintain calcium homeostasis by increasing the intestine's efficiency in absorbing dietary calcium. It helps to ensure that the amount of calcium absorbed is adequate to maintain blood calcium in the normal range and adequate to maintain skeletal mineralization. Adequate vitamin D intake facilitates intestinal absorption of calcium, and plays an important role in regulating calcium metabolism and in the mineralization of the skeleton.

Vitamin D insufficiency and deficiency are recognized as causes of metabolic bone disease in adults. Vitamin D insufficiency is characterized by the impairment of calcium and phosphate absorption but no impairment of normal bone mineralization and is typically associated with a serum 25-hydroxy vitamin D level between about ≦9 to about 30 ng/mL. Vitamin D deficiency is characterized by severely impaired calcium absorption, secondary hyperparathyroidism, hypophosphatemia, low or low normal blood calcium, and impaired bone mineralization. Serum 25-hydroxy vitamin D levels are usually about <9 ng/mL. Vitamin D insufficiency and deficiency result in increased parathyroid hormone (PTH), which in turn causes increased osteoclastic activity, urinary phosphate loss and calcium mobilization from bone. This in turn can aggravate osteoporosis, especially in older adults, as impaired bone mineralization results in independent and additional reductions in bone strength. Sustained vitamin D insufficiency is thought to be an important cause of gradual bone loss. Depending on the degree of the vitamin D and calcium deficiency, the histological picture may either be one of osteomalacia, osteoporosis or a combination of the two.

The prevalence of vitamin D insufficiency and deficiency creates a need for additional vitamin D intake in the patient populations prone to, or suffering from, conditions such as osteoporosis or osteopenia and in the subjects undergoing bisphosphonate therapy for these conditions. In subjects undergoing bisphosphonate therapy, and in particular those subjects with inadequate dietary calcium intake or inadequate calcium absorption, there is a need for adequate vitamin D to facilitate bone formation and mineralization, while minimizing the potential for or occurrence of vitamin D insufficiency. Some form of increasing vitamin D intake is often used in clinical trials of bone resorption compounds and recommended on product labels and in product package circulars. However, approximately 30% of the osteoporotic patients in, for example, the United States have some degree of vitamin D insufficiency and prevalence increases with age.

Currently, subjects taking oral bisphosphonates and requiring vitamin D are advised to take two separate products at two different times. Vitamin D formulations are most commonly taken daily, while bisphosphonates may be administered daily, weekly, monthly or at longer intervals. As a result, many patients in treatment for osteoporosis or osteopenia fail to take vitamin D despite being advised to do so. Typically, vitamin D cannot be taken simultaneously with bisphosphonates, simply due to the fact that bisphosphonate absorption is so poor, and that most bisphosphonate oral dosage regimens require a 30 minute time interval between ingestion of the bisphosphonate and other substances (including but not limited to vitamin D). As a result, patient compliance with dosing regimens that require a separate administration of a vitamin D compound at some time interval either before or after bisphosphonate administration is not high. (Some bisphosphonate compounds require administration prior to ingestion of foods, and therefore any vitamin D administration would have to occur at some time interval after bisphosphonate administration). While it is possible for patients to take vitamin D before or after taking their bisphosphonate dosages, there is evidence that many patients do not do so. A 1998 marketing study showed that while 75-85% of physicians prescribing alendronate also recommended vitamin D supplementation, only 57% of osteoporotic patients actually complied.

Although vitamin D can also be administered in the form of a multi-vitamin, in the United States, for example, many over-the-counter oral vitamin D formulations are not sold in the dosage units required for dosing less frequently than daily. And, if patients self-administer vitamin D simultaneously with their bisphosphonate dosage, it is possible that the type of vitamin D administered could interfere with and further reduce bisphosphonate absorption since many vitamin D compounds formulated for osteoporotic patients contain calcium which reduces the absorption of a bisphosphonate.

The patent literature includes patents and published patent applications that disclose vitamin D₃ or vitamin D₂ or their metabolites or analogs in combination with a bisphosphonate. See, e.g., U.S. Pat. Nos. 4,230,700, 4,330,537 and 4,812,304; European Patent Nos. EP 0 381 296 and EP 0 162 510; International Patent Publication Nos. WO 90/01321, WO 92/21355, WO 01/28564, WO 01/97788 and WO 03/086415; European Patent Publication No. EP 1 051 976; Japanese Patent Publication Nos. 7-330613 and 11-60489; U.S. Patent Application Nos. U.S. 2003/0139378 A1, and U.S. 2003/0225039 A1. These patents and publications, however, do not disclose or enable a composition, product or formulation (and, most particularly, a tablet) comprising a bisphosphonate compound and a vitamin D compound that is useful for continuous oral administration at intervals, such as once weekly, that are less frequent than daily and more frequent than six months or longer. These patents and publications also do not disclose or enable treating, inhibiting, reducing or preventing osteoporosis and other conditions associated with abnormal bone resorption by administering such bisphosphonate/vitamin D compositions at intervals less frequent than daily and more frequent than six months.

As a result, there is a need for a combination product comprising a bisphosphonate compound and a vitamin D compound, including to enhance the overall efficacy of bisphosphonate treatment by helping to assure adequate vitamin D intake to facilitate calcium absorption. There is also a need for a vitamin D and bisphosphonate product to facilitate normal bone formation and mineralization while reducing or minimizing the potential for or occurrence of complications associated with vitamin D insufficiency, such as hypocalcemia and osteomalacia. There is a need for a bisphosphonate and vitamin D product to provide an amount of vitamin D nutrition to facilitate normal bone formation and mineralization in subjects undergoing bisphosphonate therapy. There is also a need for a bisphosphonate and a vitamin D combination for oral administration according to a continuous dosing schedule at dosing intervals less frequent than daily dosing and more frequent than dosing at 6 months or longer intervals. There is a need for a single product comprising vitamin D and a bisphosphonate, suitable for once-weekly dosing, to increase the convenience of vitamin D intake and to increase patient compliance with recommended vitamin D nutrition during bisphosphonate therapy. Furthermore, there is a need for methods of preparing and administering such vitamin D/bisphosphonate compositions.

### SUMMARY OF THE INVENTION

The present invention provides pharmaceutical compositions comprising a bisphosphonate compound and a vitamin D compound. Embodiments of the present invention, for example, include pharmaceutical compositions comprising a bisphosphonate compound, or pharmaceutically acceptable salts, derivatives or hydrates of the bisphosphonate, or mixtures thereof, and a vitamin D compound, such as a pharmaceutical grade vitamin D compound. In embodiments, the vitamin D compound comprises cholecalciferol. In embodiments, the bisphosphonate comprises, for example, alendronate, a pharmaceutically acceptable salt of alendronate (for example, sodium, potassium, calcium, magnesium, or ammonium, or a hydrate of any of those salts), such as alendronate monosodium, alendronate monosodium monohydrate, or alendronate monosodium trihydrate. In an embodiment of the present invention, the pharmaceutical composition comprises cholecalciferol and alendronate monosodium trihydrate. (See FIG. 2) Compositions of the present invention may be in the form of compressed, coated or uncoated tablets, capsules, elixirs, emulsions, or other acceptable dosage forms.

In embodiments of compositions of the present invention, the bisphosphonate (or pharmaceutically effective salts, derivatives or hydrates thereof, or mixtures thereof) is present in pharmaceutically effective amounts, for example from about 0.05 mg to about 1120 mg. In the same or other embodiments of the compositions of the present invention, the vitamin D compound is present in pharmaceutically effective amounts, for example from about 100 IU to about 60,000 IU of a vitamin D compound (40 IU of vitamin D has a mass of approximately 1 microgram). In other embodiments, the present invention relates to a pharmaceutical composition comprising from about 100 IU to 36,000 IU of a vitamin D compound, and from about 5 mg to about 560 mg, on a bisphosphonic acid active basis, of a bisphosphonate, pharmaceutically acceptable salts, derivatives or hydrates thereof, or mixtures thereof. In other embodiments, the present invention relates to a pharmaceutical composition comprising from about 100 IU to 28,000 IU of a vitamin D compound, and from about 5 mg to about 280 mg, on a bisphosphonic acid active basis, of the bisphosphonate, pharmaceutically acceptable salts, derivatives or hydrates thereof, or mixtures thereof. In other embodiments, the present invention relates to a pharmaceutical composition comprising from about 100 IU to 8,400 IU of a vitamin D compound, and from about 5 mg to about 280 mg, on a bisphosphonic acid active basis, of the bisphosphonate, pharmaceutically acceptable salts, derivatives or hydrates thereof, or mixtures thereof. In other embodiments, the present invention relates to a pharmaceutical composition comprising from about 100 IU to 5,600 IU of a vitamin D compound, and from about 5 mg to about 280 mg, on a bisphosphonic acid active basis, of the bisphosphonate, pharmaceutically acceptable salts, derivatives or hydrates thereof, or mixtures thereof. In other embodiments, the present invention relates to a pharmaceutical composition comprising from about 100 IU to 4,200 IU of a vitamin D compound, and from about 5 mg to about 280 mg, on a bisphosphonic acid active basis, of the bisphosphonate, pharmaceutically acceptable salts, derivatives or hydrates thereof, or mixtures thereof. An embodiment of the present invention is a pharmaceutical composition comprising about 2,800 IU of a vitamin D and about 70 mg, on an bisphosphonic acid active basis, of a bisphosphonate or pharmaceutically acceptable salts, derivatives or hydrates of a bisphosphonate, or mixtures thereof. An embodiment of the present invention is a pharmaceutical composition comprising about 2,800 IU of cholecalciferol and about 70 mg, on an alendronic acid active basis, of alendronate or pharmaceutically acceptable salts, derivatives or hydrates of alendronate, or mixtures thereof. In a further embodiment, the cholecalcifeol is pharmaceutical grade.

An example of a composition of the present invention is a tablet comprising alendronate sodium, cholecalciferol or cholecalciferol granules containing an appropriate equivalent amount of cholecalciferol and additional excipients, such as suitable fillers, diluents, binders, lubricants, glidants, disintegrants and the like. A further example of a composition of the present invention is a tablet comprising alendronate sodium, cholecalciferol or cholecalciferol granules containing an appropriate equivalent amount of cholecalciferol, lactose, lactose anhydrous, microcrystalline cellulose, colloidal silicon dioxide, croscarmellose sodium, and magnesium stearate. Compositions of the present invention may be formulated to meet various purity and stability criteria, for example, to comprise less than 1% by weight of each isomer of cholecalciferol (relative to total cholecalciferol), after storage for 24 months at about <30° C. and about <30% relative humidity. Compositions of the present invention may also be formulated to comprise less than about 5% total degradants of cholecalciferol after storage for 24 months at about <30° C. and about <30% relative humidity. For storage purposes, the effect that environmental humidity may have on the purity and stability of the composition can be eliminated by using appropriate packaging, such as aluminum foil blister packs or HDPE bottles with dessicant.

In addition, the present invention encompasses methods of manufacturing compositions and dosage forms disclosed in this specification, as well as products made according to those methods. An embodiment of such a method comprises preparing a powder blend comprising a bisphosphonate, such as alendronate, compacting the powder blend to form a mixture, milling and blending the mixture with a vitamin D compound to form a final granule blend, and compressing the final granule blend, for example, to form tablets. In a further embodiment, the final granule blend may be lubricated before it is compressed. In an embodiment, the powder blend comprises alendronate, colloidal silicon dioxide, lactose anhydrose, microcrystalline cellulose, croscarmellose sodium and magnesium stearate. In another embodiment, the roller compacting of the powder blend forms compacted ribbons, which may be milled, blended with cholecalciferol granules, lubricated, and compressed into a solid dosage form. The advantages of the methods of manufacturing of the present invention include increased stability of the vitamin D compound in a bisphosphonate/vitamin D composition, as well as increased uniformity of the particle size of the individual compounds used in the compositions of the present invention.

Additionally, the alendronate powder blend may be pre-blended with one or more of the excipients first, blended with the rest of the excipients, and then roller compacted.

Other methods of manufacturing bisphosphonate granules, such as, for example alendronate granules, include but are not limited to, slugging, as well as wet granulation methods. If the bisphosphonate granules are manufactured using slugging, then the bisphosphonate (such as alendronate) powder blend may be compressed into a non-ribbon compact and then milled into granules, which may then be blended with a vitamin D compound to form a granule blend, which may then be compressed into a solid dosage form (e.g., a tablet). Alternatively, the bisphosphonate and all of the excipients may be wet granulated with a granulating liquid (e.g., water), then dried, milled, blended with a vitamin D compound, and processed to a final dosage form (e.g., a tablet).

In addition to the above mentioned methods, a direct blend method can also be employed by blending the bisphosphonate, all of the excipients, and the vitamin D compound together and then compressing into a tablet or encapsulting into a capsule or other solid dosage forms. Further description of possible methods for manufacturing the bisphosphonate granule are described in U.S. Pat. No. 5,358,941; U.S. Pat. No. 5,882,656 and PCT Publication WO 95/29679.

It is also possible to manufacture the bisphosphonate/vitamin D composition as described above and then perform a drying step in order to reduce the moisture level of the composition. Additionally, it is possible to package the composition with a dessicant in order to reduce the moisture level.

The present invention also encompasses methods for preventing, reducing, inhibiting or treating metabolic bone diseases. Metabolic bone diseases include, but are not limited to, osteoporosis, post-menopausal osteoporosis, steroid-induced osteoporosis, male osteoporosis, other disease-induced osteoporosis, idiopathic osteoporosis, and glucocorticoid-induced osteoporosis. The present invention also encompasses methods for preventing, reducing, inhibiting or treating osteoporosis, conditions associated with osteoporosis, and other diseases and conditions associated with abnormal bone resorption. Such other diseases and conditions may include, as further examples, metastatic bone disease, hypercalcemia of malignancy, periprosthetic osteolysis, inflammatory arthritis, and other diseases and conditions identified herein in a human or other mammal. Additionally, the present invention relates to a method for eliciting a disease modifying effect on an arthritic condition in a mammal which comprises administering to the mammal a therapeutically effective amount of a vitamin D/bisphosphonate composition. The present invention also relates to methods for eliciting a disease modifying effect on subchondral bone sclerosis, preventing osteophyte formation or progression and preventing joint destruction in a mammal, which comprise administering to the mammal a therapeutically effective amount of a vitamin D/bisphosphonate composition. The present invention also encompasses a method for reducing the risk of bone fractures in a mammal which comprises administering a unit dosage of the vitamin D/bisphosphonate composition.

Embodiments of such methods encompass administering the compositions of the present invention to mammals, including humans. Such compositions may be administered at intervals of once-weekly, bi-weekly, monthly, twice-monthly, and bimonthly. In such methods, vitamin D is provided by compositions of the present invention during bisphosphonate therapy while minimizing the occurrence of or potential for the complications associated with vitamin D insufficiency. Accordingly, compositions and methods of the present invention may be useful in mammals identified as having or being susceptible to vitamin D insufficiency or deficiency, or desiring adequate amounts of vitamin D. In an embodiment, once-weekly dosing to treat osteoporosis or another disease or condition associated with abnormal bone resorption and to minimize the risk or complications from vitamin D insufficiency, is maintained on a continuous schedule until the desired therapeutic effect is achieved. An embodiment of the methods of the present invention includes administering once weekly, to a mammal suffering from osteoporosis, a tablet comprising about 2,800 IU cholecalciferol and about 70 mg alendronate or pharmaceutically acceptable salts, derivatives or hydrates of alendronate, or mixtures thereof. In embodiments, the therapeutic effect of once-weekly administration of the vitamin D compound of a composition of the present invention is substantially similar to the therapeutic effect of a recommended daily dosage of vitamin D, for example, 400 IU, 600 IU or 800 IU vitamin D daily.

The present invention additionally encompasses methods for measuring cholecalciferol in the pharmaceutical compositions (e.g., stability) comprising cholecalciferol and a bisphosphonate. An embodiment of such a method comprises extracting cholecalciferol from such a composition into a first solution to form a second solution, separating a sample containing cholecalciferol from the second solution, and detecting an amount of the cholecalciferol in the sample, for example, using reverse-phase high performance liquid chromatography. Embodiments of such methods provide increased measurement sensitivity and may advantageously be used with compositions of the present invention to distinguish between cholecalciferol and pre-cholecalciferol, or between isomers of cholecalciferol and pre-cholecalciferol, or to detect cholecalciferol or pre-cholecalciferol ester adducts.

The present invention further encompasses methods for measuring cholecalciferol in plasma after administration of the bisphosphonate/cholecalciferol compositions of the present invention. An embodiment of such a method comprises administering to a mammal a composition comprising alendronate and cholecalciferol, obtaining from the mammal a plasma sample, extracting the cholecalciferol from the plasma sample to form a first solution, reacting the cholecalciferol in the first solution with a dienophile to form one or more diels-alder addition products of cholecalciferol, separating the diels-alder addition products of cholecalciferol using high performance liquid chromatography (HPLC) separation, and detecting an amount of cholecalciferol in the sample using mass spectroscopy. Embodiments of such methods provide an increased measurement sensitivity and may advantageously be used with the compositions of the present invention, for example, to measure the pharmacokinetic effects of administration of the compositions of the present invention.

The present invention also provides methods of measuring the pharmacokinetic effect over time of administering the compositions of the present invention, including, for example, as reflected by the total urinary excretion, area under the serum-concentration-versus-time curve (AUC), steady state maximum plasma concentration (Cₘₐₓ), time of Cₘₐₓ (Tₘₐₓ), and plasma concentration median apparent half-life (t_{1/2}) of a tablet comprising about 70 mg alendronate and about 2,800 IU cholecalciferol.

The present invention can comprise, consist of, or consist essentially of the essential as well as optional ingredients, components, steps and methods described or claimed herein. Further features, advantages and embodiments of the invention, its nature and various advantages, will become more apparent from the following detailed description, and from practice of the invention.

### BRIEF DESCRIPTION OF THE DRAWINGS

FIG. 1 depicts the metabolism of vitamin D₃.

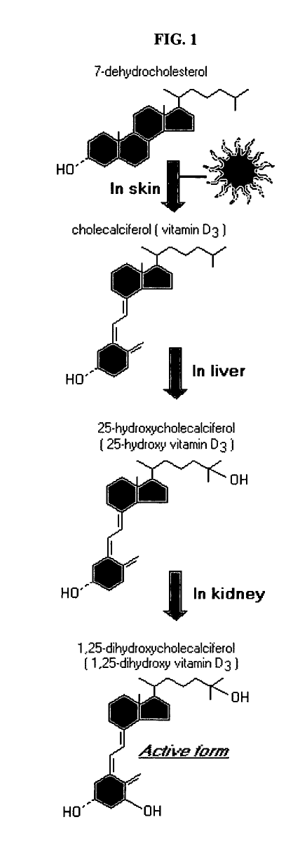

FIG. 2 depicts the chemical structures of cholecalciferol and alendronate monosodium trihydrate.

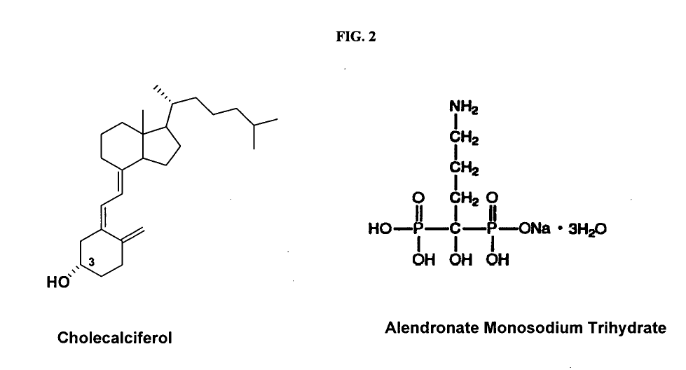

FIG. 3 depicts the chemical structures of vitamin D₂ and of vitamin D₃.

FIG. 4 shows a schematic diagram summarizing an embodiment of a method of preparing compositions of the present invention.

FIG. 5 depicts thermal and photochemical isomerizations and transesterifications of vitamin D₃.

FIG. 6 shows the results of a radiolabel study of vitamin D₃ degradation.

### DETAILED DESCRIPTION OF THE INVENTION

The term “abnormal bone resorption,” as used herein, means a degree of bone resorption that exceeds the degree of bone formation, either locally, or in the skeleton as a whole, or alternatively, can be associated with the formation of bone having an abnormal structure.

“Arthritic condition” or “arthritic conditions” refers to a disease wherein lesions, some of which are inflammatory, are confined to the joints or any inflammatory conditions of the joints, most notably rheumatoid arthritis. (Academic Press Dictionary of Science Technology; Academic Press; 1st edition, Jan. 15, 1992). An arthritic condition can be caused by inflammation, trauma or infection. The compositions of the present invention are also useful, alone or in combination, to treat or prevent arthritic conditions or symptoms/diseases involving arthritis, such as amyloidosis; ankylosing spodylitis; bacterial arthritis; basic calcium phosphate crystal deposition disease; Behcet's disease; bursitis and tendinitis; CPPD deposition disease; calcific tendonitis; carpal tunnel syndrome; Ehlers-Danlos syndrome; enteropathic arthritis; Felty's syndrome; fibromyalgia; gout; fungal arthritis; hemoglobinopathy; hemophilic arthropathy; hypertrophic osteoarthropathy; infectious arthritis; inflammatory bowel disease; juvenile arthritis; juvenile rheumatoid arthritis; lupus erythematosus; lyme disease; marfan syndrome; mixed connective tissue disease; multicentric reticulohistocytosis, myopathies; myositis; osteoarthritis; osteonecrosis; osteonecrosischondrodystrophy; polyarteritis; polymyalgia rheumatica; psoriatic arthritis; Raynaud's phenomenon; reflex sympathetic dystrophy syndrome; Reiter's syndrome; relapsing polychondritis; rheumatoid arthritis; rheumatic fever; sarcoidosis; septic arthritis; scleroderma; Sjogren's syndrome; spondyloepiphyseal dysplasia; systemic lupus erythematosus; and viral arthritis. Unlike rheumatoid arthritis, osteoarthritis is a connective tissue disease, with pathology arising from mechanical insult-induced articular cartilage degeneration, subchondral bone remodeling and limited synoviticc inflammation response. The net outcome of these activities is joint deformity secondary to erosion of articular cartilage, peri-articular endochondral ossification/osteophytosis, subchondral bone sclerosis and cyst formation. See, Oettmeier, R., and K. Abendroth, 1989, “Osteoarthritis and bone: osteologic types of osteoarthritis of the hip,” Skeletal Radiol. 18:165-74; Cutolo M, Seriolo B, Villaggio B, Pizzorni C, Craviotto C, Sulli A. Ann. N.Y. Acad. Sci. 2002 June; 966:131-42; Cutolo, M. Rheum Dis Clin North Am 2000 November; 26(4):881-95; Bijlsma J W, Van den Brink H R. Am J Reprod Immunol 1992 October-December; 28(3-4):231-4; Jansson L, Holmdahl R.; Arthritis Rheum 2001 September; 44(9):2168-75; and Purdie D W. Br Med Bull 2000; 56(3):809-23; See also Merck Manual, 17th edition, pp. 449-451. An embodiment of the present invention encompasses the treatment, reduction, inhibition or prevention of an arthritic condition which comprises administering a therapeutically effective amount of a composition of the present invention. Another embodiment is the treatment, reduction, inhibition or prevention of osteoarthritis which comprises administering a therapeutically effective amount of a composition of the present invention.

The term “bisphosphonate,” as used herein, corresponds to the chemical formula: 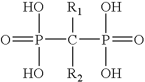
where R₁ is independently selected from the group consisting of H, OH, and Cl, R₂ is independently selected from CH₃, Cl, CH₂CH₂NH₂, (CH₂)₃NH₂, CH₂-3-pyridyl, CH₂—S-phenyl-Cl, CH₂CH₂N(CH₃)(pentyl), CH₂-imidazole, CH₂-2-imidazo-pyridinyl, N-(cycloheptyl), CH₂CH(CH₃)₂, (CH₂)₅NH₂, and CH₂-1-pyrrolidinyl, and combinations thereof. In embodiments of the present invention, R₁ is OH and R₂ is a 3-aminopropyl moiety, so that the resulting compound is a 4-amino-1-hydroxybutylidene-1,1-bisphosphonate, i.e., alendronate.

Pharmaceutically acceptable salts, derivatives, and hydrates of the bisphosphonates are also encompassed by the compounds and methods of the present invention. Non-limiting examples of salts include those selected from the group consisting alkali metal, alkaline metal, ammonium, and mono-, di-, tri-, or tetra-C₁-C₃₀-alkyl-substituted ammonium, including sodium, potassium, calcium, magnesium, and ammonium salts. Non-limiting examples of derivatives include those selected from the group consisting of esters and amides. Also encompassed within the scope of the present invention are the various hydrates and other solvates of bisphosphonates, and pharmaceutically acceptable salts thereof. Also encompassed within the scope of the present invention are hydrates of alendronate, including but not limited to, hydrates with water content between about one to twelve percent, and their crystalline forms. Non-limiting examples of hydrates of alendronate and other bisphosphonates include the monohydrate, dihydrate, trihydrate, hemihydrate, ¼ hydrate, ⅓ hydrate, ⅔ hydrate, ¾ hydrate, 5/4 hydrate, 4/3 hydrate, and 3/2 hydrate.

Non-limiting examples of bisphosphonates useful in the present invention include the following:

Alendronic acid, 4-amino-1-hydroxybutylidene-1,1-bisphosphonic acid.

Alendronate (also known as alendronate sodium or monosodium trihydrate, or by the trademark FOSAMAX®, 4-amino-1-hydroxybutylidene-1,1-bisphosphonic acid monosodium trihydrate. (Alendronic acid and alendronate are described, for example, in U.S. Pat. No. 4,922,007, to Kieczykowski et al., issued May 1, 1990, and U.S. Pat. No. 5,019,651, to Kieczykowski, issued May 28, 1991).

Cycloheptylaminomethylene-1,1-bisphosphonic acid, YM 175, Yamanouchi (incadronate or cimadronate), as described, for example, in U.S. Pat. No. 4,970,335, to Isomura et al., issued Nov. 13, 1990.

1,1-dichloromethylene-1,1-diphosphonic acid (clodronic acid), and the disodium salt (clodronate, Procter and Gamble), are, for example, described in Belgium Patent 672,205 (1966) and J. Org. Chem 32, 4111 (1967).

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described, for example, in U.S. Pat. No. 4,927,814, issued May 22, 1990.

[1-hydroxy-2-imidazopyridin-(1,2-a)-3-ylethylidene]-bis-phosphonate (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described, for example, in U.S. Pat. No. 4,761,406.

1-hydroxy-2-(3-pyridinyl)-ethylidene-1,1-bisphosphonic acid (risedronate).

(4-chlorophenyl)thiomethane-1,1-disphosphonic acid (tiludronate) as described, for example, in U.S. Pat. No. 4,876,248, to Breliere et al., Oct. 24, 1989.

1-hydroxy-2-(1H-imidazol-1-yl)ethylidene-1,1-bisphosphonic acid (zoledronate).

In embodiments of the present invention, the bisphosphonate is selected from the group consisting of alendronate, pharmaceutically acceptable salts, derivatives and hydrates thereof, and mixtures thereof. The pharmaceutically acceptable salt of alendronate may be selected from the group consisting of the sodium, potassium, calcium, magnesium, and ammonium salt of alendronate, and may be alendronate monosodium or a hydrate thereof, including for example alendronate sodium monohydrate or alendronate sodium trihydrate.

In an embodiment, the compositions of the present invention comprise alendronate sodium (monosodium salt of 4-amino-1-hydroxybutylidene-1,1-bisphosphate), which is a member of the nitrogen-containing bisphosphonate class of drugs.

It should be noted that the terms “bisphosphonate” and “bisphosphonates,” as used herein in referring to the therapeutic agents of the present invention, are meant to also encompass diphosphonates, bisphosphonic acids, and diphosphonic acids, as well as salts, derivatives and hydrates of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. Bisphosphonate doses calculated on the basis of their salt, derivative or hydrate forms are included within the dosage ranges of the present invention on the basis of their bisphosphonic acid active weights. Additionally, the doses of all hydrate forms of alendronate are calculated on the basis of the alendronic acid active weight. For instance, the doses of the monohydrate, trihydrate, hemihydrate and all other hydrate forms of alendronate and its salts, are calculated on the basis of their alendronic acid active weights. As another example, the phrase “about 70 mg of a bone resorption inhibiting bisphosphonate selected from the group consisting of alendronate, pharmaceutically acceptable salts, derivatives and hydrates thereof, and mixtures thereof, on an alendronic acid active weight basis” means that the amount of the bisphosphonate compound selected is calculated based on 70 mg of alendronic acid.

As used throughout this specification and claims, the terms “bisphosphonic acid” and “alendronic acid” include the related bisphosphonic acid forms, pharmaceutically acceptable salt forms and equilibrium mixtures of these. The terms include crystalline, hydrated crystalline, and amorphous forms of alendronic acid and pharmaceutically acceptable salts thereof. The term “alendronic acid” specifically includes, but is not limited to, anhydrous alendronate monosodium, alendronate monosodium hemihydrate, alendronate monosodium monohydrate, alendronate monosodium trihydrate, anhydrous alendronate dipotassium, and alendronate dipotassium pentahydrate. Alendronate monosodium monohydrate and other crystalline forms of alendronate sodium are disclosed in U.S. Pat. No. 6,281,381. Potassium salts of alendronic acid, and hydrates thereof, are disclosed in International Patent Publication WO 99/20635.

While it is conventional to dose and calculate the dosages of bisphosphonates on the basis of bisphosphonic acid active weight, bisphosphonate dosages can be calculated and administered based on other salt or hydrate forms. For example, dosages of the bisphosphonate risedronate are calculated based on the weight of the anhydrous risedronate sodium salt. According to the Physician's Desk Reference (55^{th} Edition, page 2664, (2001)), for example, each tablet of risedronate contains the equivalent of 5 mg or 30 mg of anhydrous risedronate sodium, in the form of the hemi-pentahydrate with small amounts of monohydrate.

The term “cholecalciferol granules” as used herein refers to the granules that contain cholecalciferol and may also contain pre-vitamin D₃, isomers of vitamin D₃, transesterfied vitamin D₃ or its isomers and/or additional excipients.

The terms “continuous schedule” or “continuous dosing schedule,” as used herein, mean that the dosing regimen is repeated until the desired therapeutic effect is achieved. The continuous schedule or continuous dosing schedule is distinguished from cyclical or intermittent administration.

The term “Dry Vitamin D₃ 100 granules,” as used herein, refers to Dry Vitamin D₃ 100, Gelatin Coated, Pharmaceutical Grade granules which are sold commercially by BASF.

The term “generalized bone loss,” as used herein, means bone loss at multiple skeletal sites or throughout the skeletal system. The term “localized bone loss,” means bone loss at one or more specific, defined skeletal sites.

The terms “human in need of treatment,” “human in need of prevention,” “human in need thereof,” and “human at risk thereof,” as used herein, refer to a human in need of treatment for a disease condition, in need of prevention, mitigation, inhibition or reduction of a disease condition, or at risk of developing a disease condition, as determined by a clinician or researcher.

The term “IU,” as used herein, means International Units. It is customary to use International Units (IU) when stating the potency and dosage of vitamin D. One International Unit (IU) is defined as the specific biologic activity of 0.025 μg of the crystalline international standard or pure vitamin D. Stated in another way, one microgram of vitamin D is approximately 40 International Units.

The terms “mammal in need of treatment,” “mammal in need of prevention,” “mammal in need thereof,” and “mammal at risk thereof,” as used herein, refer to a mammal in need of treatment for a disease condition, in need of prevention, mitigation, inhibition or reduction of a disease condition, or at risk of developing a disease condition, as determined by a clinician or researcher.

“Once-weekly dosing,” as used herein, means that a unit dosage, for example a unit dosage of bisphosphonate and a vitamin D compound, is administered once a week, i.e., once during a seven-day period, preferably on the same day of each week. In the once-weekly dosing regimen, the unit dosage is generally administered about every seven days. A non-limiting example of a once-weekly dosing regimen would entail the administration of a unit dosage of the bisphosphonate and a vitamin D compound every Sunday. It is customarily recommended that a unit dosage for once-weekly administration is not administered on consecutive days, but the once-weekly dosing regimen can include a dosing regimen in which unit dosages are administered on two consecutive days falling within two different weekly periods.

“Osteophyte” as used herein refers to newly formed bony structures located at the joint margins, and their occurrence is strongly associated with the late stage of osteoarthritis progression. The current hypothesis is that osteophytes originate from activated periosteum leading to new cartilaginous outgrowths that eventually turns into bone by the process of endochondral bone formation.

“Pharmaceutically acceptable” as used herein with reference to salts, esters, hydrates and derivates of a bisphosphonate (such as alendronate) means that the salts, derivatives or hydrates of the bisphosphonate has the same general pharmacological properties as the free acid form from which they are derived and are acceptable from a toxicity viewpoint.

The term “pharmaceutically effective amount,” as used herein, means that amount of a compound, for example a bisphosphonate compound or a vitamin D compound, that will elicit a desired therapeutic effect or response when administered in accordance with a treatment regimen. A pharmaceutically effective amount of bisphosphonate, for example, is an amount administered according to a treatment regimen that is sufficient to elicit prevention, reduction, inhibition or treatment of abnormal bone resorption, for instance.

The term “pharmaceutical grade,” as used herein, means of a sufficient quality and potency so as to conform to applicable United States Pharmacopoeia (USP) and European Pharmacopoeia (Ph. Eur.) compendial requirements. While at this time there is no USP monograph for a formulated vitamin D₃ product, an applicable Ph.Eur. monograph has been published. A “pharmaceutical grade” cholecalciferol, for example, generally is of a superior grade than the vitamin D commonly used in nutritional supplements.

The terms “preventing, inhibiting, reducing or treating,” as used herein, include addressing abnormal bone resorption (and the resultant physiological conditions—e.g., osteoporosis) through the direct or indirect alteration of osteoclast formation or activity, and encompass prevention, inhibition, reduction or treatment of bone loss, especially the inhibition of removal of existing bone either from the mineral phase and/or the organic matrix phase, through direct or indirect alteration of osteoclast formation or activity. These terms also mean addressing other disease states or conditions in such fashion as to promote relief from the disease or condition.

The term “until the desired therapeutic effect is achieved,” as used herein, means that a composition, for example, a bisphosphonate and cholecalciferol composition, is administered according to a chosen dosage schedule, or a course of therapy is followed, up to the time that the clinical or medical effect sought for the disease or condition is observed by the clinician or researcher. For methods of treatment of the present invention, the bisphosphonate compound may be continuously administered until the desired change in bone mass or structure is observed. In such instances, achieving an increase in bone mass, preventing further reduction in bone mass or replacing abnormal bone structure with more normal bone structure, are among the desired objectives. For methods of the present invention, the bisphosphonate compound may be continuously administered for as long as necessary to prevent the undesired condition. In such instances, maintenance of bone mass density is often an objective. Non-limiting examples of administration periods can range from about 2 weeks to the remaining life span of the mammal. For humans, administration periods can range from about 2 weeks to the remaining life span of the human, preferably from about 2 weeks to about 40 years, more preferably from about 1 month to about 35 years, more preferably from about 6 months to about 30 years, and most preferably from about 1 year to about 20 years.

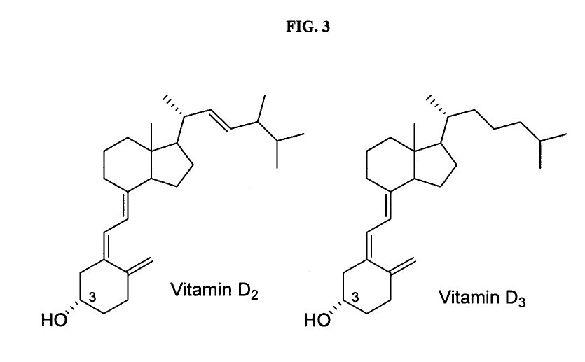

The term “vitamin D,” as used herein, means both vitamin D₂ and vitamin D₃ which have the chemical structures shown in FIG. 3. The phrase “metabolites of vitamin D” and “derivatives of vitamin D,” as used herein, mean metabolites and derivatives of vitamin D₂ and vitamin D₃. The term “vitamin D compound,” as used herein, means vitamin D₂ (ergocalciferol) and vitamin D₃ (cholecalciferol), 7-dehydrochlosterol and pre-vitamin D₂ and pre-vitamin D₃, as well as isomers of or esters of any of 7-dehydrocholesterol, vitamin D₂, vitamin D₃, pre-vitamin D₂, or pre-vitamin D₃, or mixtures thereof. At relevant physiological temperatures, vitamin D₂ and vitamin D₃ are at equilibrium with their respective pre-vitamin isomers, although that equilibrium is shifted in favor of vitamin D₂ and vitamin D₃. In the present invention, the term “vitamin D compound” does not include metabolites of vitamin D, such as, for example, 25-hydroxycholecalciferol or calcitriol or their analogs, nor does the term include the active hormone calcitriol or its analogs. The terms vitamin D₃ and cholecalciferol, are used interchangeably herein, unless expressly otherwise indicated.

The present invention provides compositions comprising a bisphosphonate, or pharmaceutically acceptable salts, derivatives or hydrates of the bisphosphonate, or mixtures thereof, and a vitamin D compound. In an exemplary embodiment, the bisphosphonate compound is selected from alendronate sodium, alendronate sodium monohydrate or alendronate sodium trihydrate, and the vitamin D compound is cholecalciferol.

The precise dosage of the bisphosphonate and the vitamin D compound will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance, but can be readily determined by the caregiver or clinician. Appropriate amounts can be determined by routine experimentation from animal models and human clinical studies. Generally, a pharmaceutically effect amount of bisphosphonate is chosen according to a continuous dosing schedule until the desired therapeutic effect is achieved. For humans, an effective oral dose of bisphosphonate is typically from about 0.0001 mg/kg to about 100 mg/kg body weight and preferably about 0.0005 to about 20 mg/kg of body weight for a 75 kg subject.

In embodiments of the present invention, an appropriate amount of the vitamin D compound is chosen to provide adequate vitamin D nutrition during the dosing interval without interfering with the bisphosphonate's ability to obtain a bone resorption inhibiting effect. For oral compositions of the present invention comprising alendronate, pharmaceutically acceptable salts, derivatives or hydrates of alendronate, or mixtures thereof, and a vitamin D compound, an amount of the vitamin D compound comprises from about 100 IU to about 60,000 IU. Non-limiting examples of an oral amount of the vitamin D compound in embodiments of the present invention include, but are not limited to, dosages of 700, 1,400 IU, 2,800 IU, 4,200 IU, 5600 IU, 7,000 IU, 8,400 IU, 14,000 IU, 28,000 IU, 36,000 IU and 60,000 IU of the vitamin D compound.

For oral compositions comprising a vitamin D compound and a pharmaceutically effective amount of alendronate, or pharmaceutically acceptable salts, derivatives or hydrates of the alendronate, or mixtures thereof, an oral pharmaceutically effective amount of alendronate typically comprises from about 0.05 mg to about 1120 mg of the alendronate compound, on an alendronic acid weight basis. Non-limiting examples of an oral pharmaceutically effective amount of alendronate in embodiments of the present invention include, but are not limited to, dosages of about 2.5 mg, 5 mg, 8.75 mg, 10 mg, 17.5 mg, 35 mg, 40 mg, 70 mg, 140 mg, 280 mg, 560, and 1120 mg of alendronate, each on an alendronic acid weight basis.

A bisphosphonate and vitamin D composition of the present invention is typically administered in admixture with suitable pharmaceutical diluents, excipients, or carriers, suitably selected with respect to a dosage form for oral administration. Examples of oral dosage forms include tablets (including compressed, coated or uncoated), capsules (each of which includes sustained release or timed release formulations), hard or soft gelatin capsules, pellets, pills, powders, granules, elixirs, tinctures, slurries, effervescent compositions, films, sterile solutions or suspensions, syrups and emulsions and the like. Likewise, it may also be administered in intravenous (bolus or infusion), intraperitoneal, topical (e.g., ocular eyedrop), intranasal, inhaled, subcutaneous, intramuscular or transdermal (e.g., patch) form, metered aerosol or liquid sprays, drops, ampoules, auto-injector devices or suppositories all using forms well known to those of ordinary skill in the pharmaceutical arts. An effective but non-toxic amount of the compositions desired can be employed. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

For example, for oral administration in the form of a tablet, capsule, pellet, or powder, the active ingredients can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water and the like. Moreover, when desired or necessary, suitable binders, fillers, diluents, lubricants, compression aids, disintegrants, buffers, coatings, and coloring agents can also be incorporated. Suitable binders can include but are not limited to starch, gelatin, natural sugars such as glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms can include but are not limited to sodium oleate, sodium stearate, magnesium stearate, sodium benzoate, sodium acetate, sodium chloride and the like. Suitable disintegrants may be one of several modified starches or modified cellulose polymers, including crosscarmellose sodium. Diluents, which may be used as compression aids, include, but are not limited to, lactose, dicalcium phosphate, cellulose, microcrystalline cellulose, and the like. Glidants, which improve the flow characteristics of a powder mixture, may also be utilized in the present invention. Examples of glidants include, but are not limited to, colloidal silican dioxide, talc, and the like. The compositions used in the present method can also be coupled with soluble polymers as targetable drug carriers. Such polymers can include but are not limited to polyvinylpyrrolidone, pyran copolymer, polyhydroxylpropyl-methacrylamide, and the like. Additional excipients, such as those described in U.S. Pat. No. 5,358,941; U.S. Pat. No. 5,882,656 and PCT Publication WO 95/29679, may also be utilized.

An embodiment of the present invention, for example, is a 80 mg to 1500 mg tablet including about 0.5% to about 90% alendronate sodium by weight, about 1% to about 70% cholecalciferol granule by weight (equivalent to about 0.0005% to about 20% cholecalciferol by weight), about 10% to about 80% lactose anhydrous by weight, about 5% to about 50% microcrystalline cellulose by weight, about 0.1% to about 5% colloidal silicon dioxide by weight, about 0.5% to about 10% croscarmellose sodium by weight, and about 0.5% to about 5% magnesium stearate by weight.

The weight range of Dry Vitamin D₃ 100 granules is to ensure 2800 IU potency in each tablet because the granule contains a potency range of 100,000 IU to 110,000 IU vitamin D₃ per gram. The quantity of lactose anhydrous is adjusted according to the amount of Dry Vitamin D₃ 100 granules added to the tablet in order to maintain a final tablet weight of 325 mg. Other non-limiting examples of oral compositions of the present invention comprising a bisphosphonate compound, such as alendronate, and a vitamin D compound, are described herein, including in the Examples below. The Dry Vitamin D₃ 100 granules contain about 100,000 IU vitamin D₃ per one gram of granule weight. Thus, 28 mg of Dry Vitamin D₃ 100 granules contains about 2800 IU of vitamin D₃, which is the equivalent of about 70 μg vitamin D₃.

Bisphosphonate/vitamin D compositions of the present invention, including the embodiments described herein, may be administered at intervals of once-weekly, bi-weekly, monthly, twice monthly, and bi-monthly. For once-weekly dosing with a composition of the present invention, an oral pharmaceutically effective amount of alendronate comprises from about 0.05 mg to about 1120 mg of the alendronate compound, on an alendronic acid active weight basis. Embodiments of the present invention providing a weekly oral pharmaceutically effective amount of alendronate include, but are not limited to, unit dosages which are useful for preventing osteoporosis comprising a vitamin D compound and from about 35 mg to about 70 mg of the alendronate compound; a unit dosage which is useful for treating osteoporosis comprising a vitamin D compound and about 70 mg of the alendronate compound; a unit dosage which is useful for treating Paget's disease comprising a vitamin D compound and about 280 mg of the alendronate compound; and a unit dosage which is useful for treating metastatic bone disease comprising a vitamin D compound and about 280 mg of the alendronate compound.

For once-weekly dosing, a pharmaceutically effective amount of a vitamin D compound in a bisphosphonate/vitamin D composition of the present invention comprises from about 100 IU to about 60,000 IU of vitamin D. Accordingly, in an embodiment of the present invention, the composition comprises from about 100 IU to about 5,600 IU of a vitamin D compound, and a pharmaceutically effective amount of alendronate, pharmaceutically acceptable salts, derivatives or hydrates of alendronate, or mixtures thereof. In another embodiment, the pharmaceutically acceptable amount of alendronate comprises from about 0.05 mg to about 1120 mg, on an alendronic acid active basis, of alendronate, pharmaceutically acceptable salts, derivatives or hydrates of alendronate or mixtures thereof.

For bi-weekly or bimonthly dosing, a pharmaceutically effective amount of a vitamin D compound in a bisphosphonate/vitamin D composition of the present invention comprises from about 100 IU to about 60,000 IU of vitamin D. In an embodiment of the present invention, the composition comprises from about 100 IU to about 8,400 IU of a vitamin D compound, and a pharmaceutically effective amount of alendronate, pharmaceutically acceptable salts, derivatives or hydrates of alendronate, or mixtures thereof. In another embodiment, the pharmaceutically acceptable amount of alendronate comprises from about 0.05 mg to about 1120 mg, on an alendronic acid active basis, of alendronate, pharmaceutically acceptable salts, derivatives or hydrates of alendronate or mixtures thereof.

For monthly dosing, a pharmaceutically effective amount of a vitamin D compound in a bisphosphonate/vitamin D composition of the present invention comprises from about 100 IU to about 36,000 IU of vitamin D. In an embodiment of the present invention, the composition comprises from about 100 IU to about 11,200 IU of a vitamin D compound, and a pharmaceutically effective amount of alendronate, pharmaceutically acceptable salts, derivatives or hydrates of alendronate, or mixtures thereof. In another embodiment, the pharmaceutically acceptable amount of alendronate comprises from about 0.05 mg to about 1120 mg, on an alendronic acid active basis, of alendronate, pharmaceutically acceptable salts, derivatives or hydrates of alendronate or mixtures thereof.

The present invention also encompasses methods for preventing, reducing, inhibiting and treating diseases and conditions associated with abnormal bone resorption, such as osteoporosis. A person suffering from osteoporosis, i.e., has a bone mineral density (BMD) which is at least about two or two and one-half standard deviations below the norm of pre-menopausal women, would be a candidate for administration of a composition of the present invention according to a method of the present invention. It has been found that vitamin D₃, administered in a once-weekly dose up to seven or more times than the amounts that would be given on a daily basis, can be simultaneously co-administered with a bisphosphonate, such as alendronate, without adversely affecting the bioavailability of the bisphosphonate. See, e.g., Example 7. The methods of the present invention do not have disadvantages of current methods of treatment which require cumbersome, irregular, or complicated dosing regimens to provide adequate vitamin D during bisphosphonate therapy.

As a result, a composition of the present invention, such as a composition comprising a bisphosphonate compound, for example alendronate, and a vitamin D compound, would be effective for all of the indications for which compositions comprising alendronate or other bisphosphonates without a vitamin D compound are effective. The methods and compositions of the present invention are useful for reducing or inhibiting bone resorption, and for treating, reducing, inhibiting or preventing abnormal bone resorption, and conditions associated therewith. Compositions of the present invention can thus be used in humans and other animals to increase bone mass and to prevent, inhibit, reduce and treat the following conditions and disease states: bone loss; osteoporosis, including but not limited to, post-menopausal osteoporosis, steroid-induced osteoporosis, male osteoporosis, disease-induced osteoporosis, idiopathic osteoporosis, and glucocorticoid-induced osteoporosis; osteonecrosis, Paget's disease; osteoarthritis; rheumatoid arthritis, other arthritic conditions, abnormally increased bone turnover; localized bone loss associated with periprosthetic bone loss or osteolysis; bone fractures; metastatic bone disease; Gaucher's disease; avascular necrosis; polyostotic fibrous dysplasia; Charcot's joint; parasitic disorders; osteogenesis imperfecta; homocystinuria; lysinuric protein intolerance; Turner's syndrome; immobilization; fibrous dysplasia ossificans progressive; fibrogenesis imperfecta ossium; periodontal disease; tooth loss; hypercalcemia of malignancy; multiple myeloma; osteopenia, including but not limited to, immobilization-induced osteopenia and osteopenia due to bone metastases; and other bone diseases and conditions that may be associated with abnormal bone resorption.

The present invention relates to the use of a composition of the instant invention for the preparation of a medicament useful in the treatment, reduction, inhibition or prevention of an arthritic condition. The present invention also relates to the use of a composition of the instant invention and an agent selected from androgen receptor modulator; an inhibitor of osteoclast proton ATPase; an inhibitor of HMG-CoA reductase; an osteoblast anabolic agent; calcitonin; Vitamin K₂ or a pharmaceutically acceptable salts and mixtures thereof, for the preparation of a medicament useful in the treatment of an arthritic condition.

In an embodiment of the invention, the arthritic condition is amyloidosis; ankylosing spodylitis; bacterial arthritis; basic calcium phosphate crystal deposition disease; Behcet's disease; bursitis and tendinitis; CPPD deposition disease; calcific tendonitis; carpal tunnel syndrome; Ehlers-Danlos syndrome; enteropathic arthritis; Felty's syndrome; fibromyalgia; gout; fungal arthritis; hemoglobinopathy; hemophilic arthropathy; hypertrophic osteoarthropathy; infectious arthritis; inflammatory bowel disease; juvenile arthritis; juvenile rheumatoid arthritis; lupus erythematosus; lyme disease; marfan syndrome; mixed connective tissue disease; multicentric reticulohistocytosis, myopathies; myositis; osteoarthritis; osteonecrosis; osteonecrosischondrodystrophy; polyarteritis; polymyalgia rheumatica; psoriatic arthritis; Raynaud's phenomenon; reflex sympathetic dystrophy syndrome; Reiter's syndrome; relapsing polychondritis; rheumatoid arthritis; rheumatic fever; sarcoidosis; septic arthritis; scleroderma; Sjogren's syndrome; spondyloepiphyseal dysplasia; systemic lupus erythematosus; and viral arthritis.

An embodiment of the invention is a method of treating, reducing, inhibiting or preventing the progression of osteoarthritis in a mammal in need thereof, comprising administering to the mammal a therapeutically effective amount of a composition of the instant invention. It is known in the literature that osteoarthritis is accompanied with a well-defined changes in the joints, including erosion of the articular cartilage surface, peri-articular endochondral ossification/osteophytosis, and subchondral bony sclerosis and cyst formation. See Oettmeier R, Abendroth, K, “Osteoarthritis and bone: osteologic types of osteoarthritis of the hip,” Skeletal Radiol. 1989; 18: 165-74. Recently, the potential contribution of subchondral bone sclerosis to the initiation and progression of osteoarthritis have been suggested. Stiffened subchondral bone as the joint responding to repetitive impulsive loading, is less able to attenuate and distribute forces through the joint, subjecting it to greater mechanical stress across the articular cartilage surface. This in turn accelerates cartilage wear and fibrillate. See Radin, E L and Rose R M, “Role of subchondral bone in the initiation and progression of cartilage damage,” Clin. Orthop. 1986; 213: 34-40. Inhibition of excessive subarticular bone resorption by a composition of the instant invention could lead to inhibition of subchondral bone turnover, and thus may have a favorable impact on osteoarthritis progression.

Another embodiment of the invention is a method of treating, reducing, inhibiting or preventing rheumatoid arthritic conditions in a mammal in need thereof, comprising administering to the mammal a therapeutically effective amount of a composition of the instant invention. It is known in the literature that progressive destruction of the periarticular bone is a major cause of joint dysfunction and disability in patients with rheumatoid arthritis. See Goldring S R, “Pathogenesis of bone erosions in rheumatoid arthritis” Curr. Opin. Rheumatol. 2002; 14: 406-10. In addition, generalized bone loss is a major cause of morbility associated with severe rheumatoid arthritis. The frequency of hip and spinal fractures is substantially increased in patients with chronic rheumatoid arthritis. See Gould A, Sambrook, P, Devlin J et al, “Osteoclastic activation is the principal mechanism leading to secondary osteoporosis in rheumatoid arthritis,” J. Rheumatol. 1998; 25: 1282-9. The use of anti-resorptive agents in the treatment or prevention of resorption in subarticular bone and of generalized bone loss represents a rational approach for pharmacological intervention on the progression of rheumatoid arthritis. Accordingly, the compositions of the present invention comprising a bisphosphonate compound and a vitamin D compound can be used to treat, reduce, inhibit or prevent the bone loss associated with rheumatoid arthritis and other osteoarthritic conditions.

More generally, it is believed that bisphosphonates can be given in the same formulation with vitamin D without adversely affecting the bioavailability of the bisphosphonate. Furthermore, it is believed that lower doses of vitamin D and higher doses of vitamin D can be given in the same formulation with bisphosphonates without affecting their bioavailability. As an example, a once-weekly dosage of 2800 IU vitamin D is believed to be effective when administered in combination with a bisphosphonate in the compositions of the present invention. It is also known that vitamin D₂ can be used in place of vitamin D₃ with similar results as those found for vitamin D₃. Thus, administration of a vitamin D compound in the same formulation with a bisphosphonate compound eliminates the separate dosing requirements of vitamin D during bisphosphonate treatment and provides vitamin D nutrition without adversely affecting the bioavailability and efficacy of the bisphosphonate.

Patients would benefit from the vitamin D and bisphosphonate combination because it provides additional vitamin D nutrition to facilitate normal bone formation and mineralization and to enhance the efficacy of bisphosphonate treatment. From patient lifestyle and compliance standpoints, the methods of the present invention would also be more convenient than daily or cyclic dosing regimens for bisphosphonates with additional daily vitamin D administration. As a result of this invention, patients may no longer separately need to take vitamin D daily to benefit from additional vitamin D nutrition because this invention provides for once-weekly doses of vitamin D. Patients will not need to keep track of a complex dosing regimen of separate bisphosphonate and vitamin D administration. Finally, patients will be subjected less frequently to the inconvenience of having to take the bisphosphonate compounds on an empty stomach and having to fast for at least 30 minutes before or after dosing. The methods of the present invention are thus likely to have the advantage of promoting better patient compliance, and which in turn can translate into better therapeutic efficacy.

It is also believed that a bisphosphonate/vitamin D composition would also be less irritating to the esophageal, as well as the gastrointestinal system. Since alendronate could potentially penetrate into the stratum basale of the stratified squamous epithelium (e.g., via its own penetration or via penetration into a site of local injury caused by abrasive food or other agent), it could cause an inhibition of keratinocyte growth, as suggested by its effects on keratinocyte growth in vitro. See, A. A. Reszka et al., Mol. Pharmacol., 2001; 59(2):193-202. Suppression of growth could slow the process of epithelial repair, thus leading to local irritation or ulceration. Autoradiograms of rats fed radioactive 1,25(OH)₂ vitamin D₃ do show evidence of expression of the vitamin D receptor in the epithelium of the esophagus. See Stumpf, W E, et al., Histochemistry, 1987; 87(1):53-8. Levels are below those seen in the parathyroid gland.

Therefore, it is believe that coadministration of an active vitamin D₃ metabolite (e.g. 1,25(OH)₂-cholecalciferol or calcitriol) with alendronate could exacerbate esophageal irritation through its differentiative effects on keratinocytes. Both calcitriol and 25-OH-cholecalciferol have been observed to effect keratinocytes by inhibiting growth and inducing differentiation. See K. Matsumoto et al., Biochem. Biophys. Acta., 1991; 1092(3):311-8. Keratinocyte differentiation is associated with cell cycle arrest (growth arrest), and thus the combination of alendronate with an active vitamin D metabolite could have synergistic effects on inhibiting growth in the stratum basale. This could in turn cause a greater irritant effect. Because oral vitamin D₃ (cholecalciferol) requires activation in the liver and then the kidney, it is believed that it would not elicit the same local irritant effect as an active metabolite.

Additionally, it may be possible that normal physiological levels of active vitamin D₃ hormone could assist the body in its attempt to repair sites of local irritation induced by acute exposure to alendronate. Because oral vitamin D₃ is administered in combination with the alendronate, and because a large proportion of the elderly population is vitamin D₃ deficient, this may better enable the body to speed the healing process along.

It is also believed that a vitamin D/bisphosphonate composition is useful for the prevention or treatment of sway. Additionally, it is believed that a vitamin D/bisphosphonate composition is useful for reducing falls. It is believed that a vitamin D/bisphosphonate composition will increase muscle strength, improve neuromuscular function, reduce body sway and improve physical function in elderly people. This would lead to a reduced risk of falls and thus contribute towards a reduced risk of bone fractures. Epidemiologic studies demonstrate the high prevalence of vitamin D deficiency in elderly in the U.S. See A. N. Exton-Smith et al., Lancet 1966; 2:999-1001; R P Heaney et al., Osteoporos Int 2000; 11:553-5; M J McKenna, Am J Med 1992; 93:69-77; S T Haden et al., Calcif Tissue Int 1999; 64:275-9. There is evidence for the effect of vitamin D on extra-skeletal tissues. See Latham et al, 2003; 51:1219-1226. Additionally, vitamin D receptors have been identified in muscle tissue and muscle weakness, limb pain and impaired physical function are well recognized manifestations of severe vitamin D deficiency.

A number of prospective, randomized, intervention studies demonstrated the efficacy of vitamin D to improve musculoskeltal function and reduce fall risk. Treatment with vitamin D, and calcium, has been shown to reduce the incidence of non-vertebral fractures and to reduce postural sway and possibly the incidence of falls. See J. K. Dhesi et al., Age and Aging 2002; 31:267-271. Additionally, it has been demonstrated that the number of falls in elderly community-dwelling patients can be significantly reduced by treatment with alfacalcidol (1-alfa-hydroxyvitamin D₃), and minimal calcium intake. See L. Dukas et al, JAGS 2004; 52:230-236.

It is also believed that a vitamin D/bisphosphonate composition will enhance the absorption of calcium. There have been studies which utilized the active metabolites of vitamin D to examine the positive effects on fractional calcium absorption in postmenopausal women. See M. L. Holzherr et al., Osteoporosis Int 2000; 11:43-51; J. C. Gallagher et al., J. Clin. Endocrinol. Metab., 1980; 51(5): 1359-64. Bisphosphonates have also been shown to increase intestinal calcium absorption in rat models. See P. Ammann et al., J Bone Miner Res 1993; 8(12):1491-8; H. Fleisch Osteoporos Int 1996; 6:166-70; J-P Bonjour Endocrinol Metab 1988; 17:E260-E264. However, it is believed that a composition of a vitamin D compound and a bisphosphonate would increase absorption of calcium more than the additive effect of vitamin D or a bisphosphonate each administered alone. Additionally, it is believed that a composition of cholecalciferol and alendronate would greatly enhance the absorption of calcium more than a combination of an active form of vitamin D and a different bisphosphonate. This increase in calcium absorption would correlate to a reduction in fracture risk.

The present invention also provides for the use of a composition comprised of a vitamin D compound and a bisphosphonate compound comprising a pharmaceutically effective amount of at least one bisphosphonate, or a pharmaceutically acceptable salt, derivative or hydrate of the bisphosphonate, or mixtures thereof, and one or more active ingredients for the manufacture of a medicament for the treatment, reduction, inhibition or prevention, in mammals such as humans, of the conditions and disease states identified above.

In further embodiments, the methods and compositions of the present invention can also comprise a histamine H2 receptor blocker (i.e., antagonist) and/or a proton pump inhibitor, which are well known therapeutic agents for increasing gastric pH. See, e.g., L. J. Hixson, et al., Current Trends in the Pharmacotherapy for Peptic Ulcer Disease, Arch. Intem. Med., vol. 152, pp. 726-732 (April 1992). It is found in the present invention that the sequential oral administration of a histamine H2 receptor blocker and/or a proton pump inhibitor, followed by a bisphosphonate and vitamin D composition can help to minimize adverse gastrointestinal effects. In one embodiment of the present invention, the histamine H2 receptor blocker and/or proton pump inhibitor is administered from about 30 minutes to about 24 hours prior, or from about 30 minutes prior to about 12 hours prior, to the administration of the bisphosphonate and vitamin D composition. The dosage of the histamine H2 receptor blocker and/or proton pump inhibitor will depend upon the particular compound selected and factors associated with the mammal to be treated, i.e., size, health, etc. Non-limiting examples of histamine H2 receptor blockers and/or proton pump inhibitors include those selected from the group consisting of cimetidine, famotidine, nizatidine, ranitidine, omprazole, and lansoprazole.

The present invention further encompasses methods of manufacturing compositions of the present invention, including for example pharmaceutical compositions comprising a bisphosphonate compound and a vitamin D compound. In an embodiment, a method for preparing an alendronate-cholecalciferol formulation, comprises: preparing a powder blend comprising alendronate; compacting the powder blend to form an alendronate mixture; milling and blending the alendronate mixture with cholecalciferol granules to form a blend; and lubricating and compressing the blend. In another embodiment, a method for preparing an alendronate-cholecalciferol solid dosage form comprises: blending alendronate, colloidal silicon dioxide, lactose anhydrous, microcrystalline cellulose, and croscarmellose sodium to form a pre-blend; blending the pre-blend and magnesium stearate to form a first lubricated mixture; roller compacting the first lubricated mixture to form compacted ribbons; milling the compacted ribbons to form a lubricated blend; blending the lubricated blend with cholecalciferol granules to form a second lubricated mixture; and compressing the second lubricated mixture into the solid dosage form.

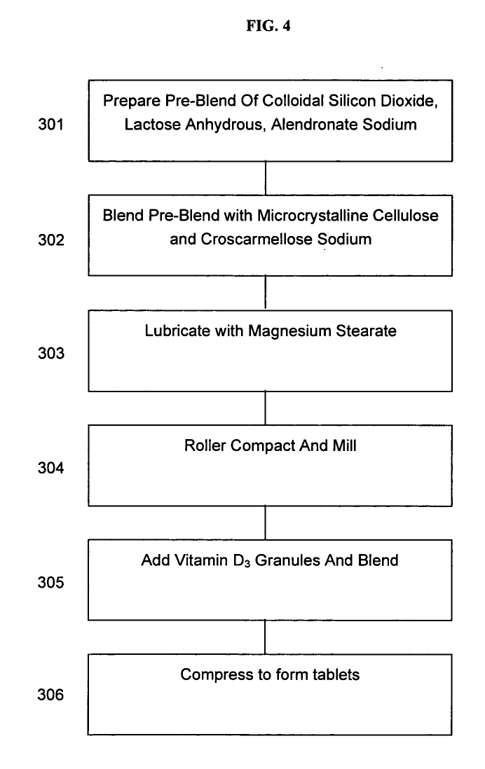

FIG. 4 depicts a flow-chart of an embodiment of a method of the present invention for manufacturing a bisphosphonate/vitamin D compositions of the present invention. In this embodiment, the composition is made by a process comprising roller compacting an alendronate sodium formulation to form a ribbon, milling of the ribbon produced from the roller compaction step and then blending with the extragranular addition of the vitamin D₃ formulation. Using, for example, the active ingredients and excipients identified in Example 1, this formulation and process results in a product which satisfies regulatory requirements for product release and stability of both alendronate and vitamin D₃. As shown in FIG. 4, in this embodiment, at step 301 a pre-blend of colloidal silicon dioxide, lactose anhydrous, and alendronate sodium is prepared. As depicted by step 302, the pre-blend is then blended with microcrystalline cellulose and croscarmellose sodium. At step 303, magnesium stearate is added to form a lubricated mixture. The lubricated mixture is passed through a roller compactor and the compacted ribbons are milled, as indicated at step 304. In the embodiment depicted in FIG. 4, at step 305 vitamin D₃ granules containing about 2800 IU (or the equivalent of about 70 μg) of vitamin D₃ are then added and blended with the milled granules, with the vitamin D₃ granule charge quantitity adjusted based on both incoming granule assay and the yield from the roller compaction/milling step. The resulting mixture is then compressed at step 306 to form tablets and the compressed tablets are de-dusted. The resulting tablets may be packaged in suitable packaging, including for example moisture-proof and light-tight blister packs or bottles.

Vitamin D₃ (cholecalciferol) and vitamin D₂ (ergocalciferol) are water insoluble, hydrophobic compounds with a melting point of about 84° C. and about 115° C., respectively. These compounds are also highly prone to oxidation and are photolabile, breaking down into various degradation products. Vitamin D granule is also prone to segregation. The stability of vitamin D is thus affected by the extent and nature of processing as well as the storage conditions (e.g., exposure to light, high temperatures, and high relative humidity) of the vitamin D/bisphosphonate compositions. As a result, the desire to include vitamin D in the compositions of the present invention presents a particular challenge insofar as developing methods of manufacturing and storing vitamin D-containing compositions is concerned. Accordingly, there is a need for vitamin D/bisphosphonate compositions that have been formulated so as to reduce the degradation of the vitamin D, both during processing and during storage. There is also a need for methods of manufacturing such stable compositions. In addition, there is a need to develop methods of detecting or measuring the degradation of vitamin D in vitamin D-containing compositions, such as those of the present invention. In addition, because the level of a particular vitamin D degradant may be very small (on the order of nanograms), there is a need to develop methods of measuring or detecting degradation of vitamin D in vitamin-D containing compositions, such as those of the present invention, having a limit of quantitation (LOQ) sufficient to detect the amounts of vitamin D degradants.

Accordingly, the present invention also provides methods of manufacturing compositions comprising a bisphosphonate compound and a vitamin D compound that minimizes the loss of the vitamin D compound during manufacture. By controlling the humidity during manufacture, temperature and light present during the formulation of vitamin D or providing the appropriate finished dosage form packaging, it is possible to reduce the loss of vitamin D while maintaining fully its potency. In an embodiment of the instant invention, the temperature during the manufacturing process is less than or equal to about 35° C. In a further embodiment, the temperature is between about 20° C. to about 30° C. In an embodiment, the relative humidity during the manufacturing process is less than or equal to about 60% RH. In another embodiment, the relative humidity is between about 20% to about 40%. In addition, controls may be placed on the starting moisture levels of not only the vitamin D components of the formulation, but also on any excipients that may be present. In a further embodiment, the relative humidity is between about 25% to about 35%.

The present invention also encompasses methods of manufacture that comprise an additional drying step. Thus, as another embodiment, the compositions of the instant invention may be manufactured under different conditions (temperature and/or relative humidity) as described above, and the moisture content of the manufactured composition may be reduced by drying the composition. In an embodiment, the drying may involve drying (with, for example, heat) the compositions of the present invention after the solid dosage form has been created. In another embodiment, the drying may involve film coating of a solid dosage forms (e.g., tablets) of the compositions of the present invention. In another embodiment, the drying may also involve packaging the compositions of the present invention with appropriate amounts of dessicants or other moieties to reduce moisture content. In another embodiment, the drying may involve storing the compositions of the present invention in storage forms that reduce moisture and/or light (e.g., aluminum blister packs, moisture-proof bottles).

In embodiments of the present invention, vitamin D compounds used as a starting material may include a free flowing, stabilized granules of vitamin D. In embodiments, the vitamin D granules used as a starting material in manufacturing methods of the present invention are Dry Vitamin D₃ 100, Gelatin Coated, Pharmaceutical Grade, sold by BASF. The particles of vitamin D are dissolved in medium chain triglycerides in droplets of 1-2 μm embedded in a starch-coated matrix of gelatin and sucrose. The dissolved vitamin D can then be stabilized with t-butylhydroxytoluene (BHT). The vitamin D granules contain sodium aluminum silicate as a flow aid. One with ordinary skill in the art would understand that the amount of vitamin D added to the composition may be need to be adjusted based the source of vitamin D and/or the potency of the vitamin D being added. For example, if Dry Vitamin D₃ 100 Gelatin Coated, Pharmaceutical Grade granules (BASF) were utilized, one with ordinary skill would understand that the granules may have different potencies (e.g., 100,000 IU/g or 105,000 IU/g or 110,000 IU/g) which would require one to adjust the amount of granules added to the composition in order to achieve 2800 IU, or 5600 IU, of vitamin D in the composition.

The vitamin D contained in an embodiment of the present invention conforms to the acceptance criteria of the Ph. Eur. Cholecalciferol Concentrate (Powder Form) monograph. While at this time there is no USP monograph for a formulated vitamin D₃ product, an applicable Ph.Eur. monograph has been published. The inactive ingredients in the compositions of the vitamin D compounds used in embodiments of compositions of the present invention (e.g., medium chain triglycerides, butylated hydroxytoluene, sucrose, gelatin, modified starch, and sodium aluminum silicate) are either compendial or food grade materials.

In embodiments of the methods and compositions of the present invention, the alendronate used as a starting material is compendial grade alendronate sodium monohydrate, or compendial grade alendronate sodium trihydrate, obtained from Merck & Co., Inc.

In addition, commercially available vitamin D granules can possibly be used in the compositions of the present invention, such as those available from Roche, BASF, or Solvay.

In further embodiments, the present invention provides a kit for conveniently and effectively carrying out the methods in accordance with the present invention. Such kits are especially suited for the delivery of solid oral forms such as tablets or capsules and in embodiments include a number of unit dosages a card having the dosages oriented in the order of their intended use. An example of such a kit is a “blister pack.” Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered. Alternatively, placebo dosages, or calcium or dietary supplements, either in a form similar to or distinct from the bisphosphonate and vitamin D unit dosages, can be included to provide a kit in which a dosage is taken every day. In those embodiments including a histamine H2 receptor and/or proton pump inhibitor, these agents can be included as part of the kit.

The present invention also provides a detection method that was developed in order to measure the degradation products of the vitamin D₃ compounds of the present invention. Specifically, a method of measuring the degradation of the pharmaceutical composition may comprise extracting the cholecalciferol from the composition into a first solution to form a second solution, separating a sample containing cholecalciferol from the second solution, and detecting the amount of cholecalciferol in the sample by subjecting the sample to reverse-phase HPLC separation. The detection method of the present invention is carried out to detect about 2800 IU to about 5600 IU cholecalciferol per pharmaceutical composition. Additionally, the detection method has a limit of quantitation (LOQ) of cholecalciferol of less than about 9 ng/mL cholecalciferol.

In an embodiment, the method utilizes a first solution which comprises water, alcohol, acetonitrile or mixtures thereof. In a specific embodiment, the first solution contains about 5% water and about 95% methanol. An exemplary sample preparation may be extracted of 15 tablets containing 2800 IU of vitamin D each into about 50 mL of about 5% water and about 95% methanol. Also in an embodiment, the resulting solution may be stirred for about 10 minutes, sonicated for about 30 minutes, and then stirred for an additional 3 hours. In an embodiment, the separating of the samples may be carried out by centrifugation, which can be from about 5,000 rpm to about 15,000 rpm. In an embodiment, the column is a Phenomenex Phenosphere 80 Å ODS (1) column (150×4.6 mm, 3 μm), and the injection volume is 100 μL. The samples are eluted down the column and then detected. In an embodiment of this method, a 65-minute gradient may be used. A detection wavelength of about 260 nm to about 265 nm may also be used. In an embodiment of this method, the detecting step is accomplished at a reverse-phase HPLC column temperature of about 25° C. A sample tray temperature of about 5° C. may be used. In an embodiment, the detecting step comprises reverse-phase HPLC separation using an eluant of about 99% acetonitrile and about 1% of 0.025% phosphoric acid.

In an embodiment, the reverse-phase HPLC column that may be used in the methods of the present invention include columns that are either only partially endcapped or not endcapped. The endcapping process reduces the free silanol groups on the stationary phase, therefore, it affects the separation between pre-vitamin D and vitamin D peaks. Attempts to use endcapped columns were unsuccessful in providing a peak resolution that was sufficient for the assay method of the present invention because any degradate eluting between the two actives would not be resolved and quantitated. Indeed, in identifying columns for use with the methods of the present invention, a vitamin D₃ isomer (0.96 RRT) was observed as eluting between two actives found in the formulation. More method development using other endcapped columns all showed limited resolution between pre-vitamin D₃ and vitamin D₃ peaks.

Column carbon loading has an impact on the elution of four vitamin D₃ ester adducts, which are the products of transesterification between either pre-vitamin D₃ or vitamin D₃ and the medium chain triglycerides (C₈ and C₁₀ fatty acid esters are present in BASF vitamin D granules that may be used in the compositions and methods of the present invention). These esters may react with the hydroxyl group of vitamin D₃ through a transesterification mechanism to form C₈-D₃ and C₁₀-D₃ esters. Because of the long fatty acid chains, these esters are very hydrophobic and interact with the C₁₈ stationary phase. A column with higher carbon loading has more C₁₈ stationary phase; therefore, it interacts more strongly with the esters and retains the esters on the column for a longer period of time. Accordingly, in an embodiment of the methods of the present invention, a HPLC column having less than about 10% carbon loading may be used. Using a column with lower carbon loading reduced the interaction between the stationary phase and the esters, resulting in earlier elution of these peaks. Results showed that, for example, using a Platinum EPS C₁₈ column with low carbon loading (5%), all esters were eluted before 10 minutes when a mobile phase containing 95% acetonitrile/5% water was used. Similarly, all four esters were eluted within 26 minutes when another column, Phenosphere ODS (1) column (7% carbon loading), was used.

Exemplary chromatographic conditions that may be used in the methods of the present invention are listed below:

| | |
|---|---|
| Flow Rate: | 1.2 mL/min |
| Column Temperature: | 25° C. |
| Injection Volume: | 100 μL |
| Mobile Phase: | Gradient, A = 0.025% phosphoric acid, |
| | B = 99% Acetonitrile/1%A |
| Run Time: | 65 minutes |
| Column: | Phenosphere 80 Å, ODS (1) column, |
| | 150 × 4.6 mm, 3 μm |
| Sample Tray Temperature | 5° C. |
| Detector Wavelength: | 265 nm |

Gradient Time Table:

| | T (min) | | | | | | |
|---|---|---|---|---|---|---|---|
| | 0 | 16 | 39 | 43 | 57 | 57.01 | 65 |
| % Aqueous | 51.5 | 13 | 10 | 0 | 0 | 51.5 | 51.5 |
| % Mixture | 48.5 | 87 | 90 | 100 | 100 | 48.5 | 48.5 |

Using detection methods of the present invention, both pre-vitamin D and vitamin D peaks can be quantitated to calculate the total amount of vitamin D in a sample. Specifically, the methods of the present invention are sufficiently sensitive and selective, with a sample of about 2800 IU cholecalciferol, to distinguish between cholecalciferol, pre-cholecalciferol, and their isomers, and to detect one or more cholecalciferol ester adducts, or one or more pre-cholecalciferol ester adducts.

Three types of potential vitamin D₃ degradation products have been observed in stability studies for the pharmaceutical compositions of the present invention, which are described below (including in Example 6). In the stability studies described below, the tablet composition that was studied comprises about 91.4 mg alendronate sodium, about 26.7 mg cholecalciferol granules, about 131.0 mg microcrystalline cellulose, about 62.4 mg lactose anhydrous, about 9.7 mg croscarmellose sodium, about 0.8 mg colloidal silicon dioxide, and about 3.1 mg magnesium stearate.

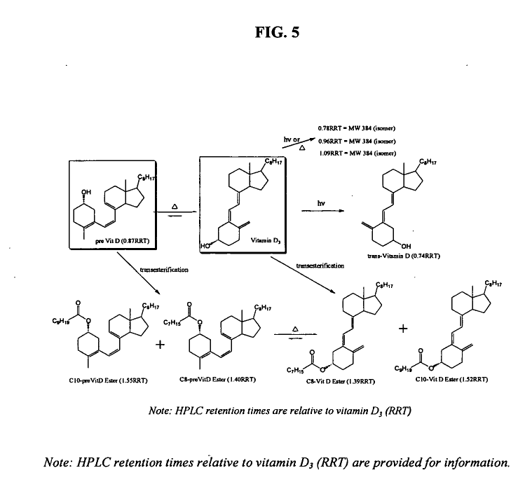

As depicted in FIG. 5, the structure of vitamin D₃ includes a conjugated triene, which undergoes a variety of thermal and photochemical isomerizations. Five vitamin D₃ isomers have been identified in exemplary pharmaceutical compositions of the present invention (in this example, using vitamin D₃ (cholecalciferol)): pre-vitamin D₃, trans-vitamin D₃, and three additional isomers at 0.78 RRT, 0.96 RRT and 1.09 RRT (which is a measure of retention time of the compound by high performance liquid chromatography (HPLC) as described below). Structures for some of these vitamin D₃ isomers are shown in FIG. 5. Structural conclusions are based on UV, MS, and in some cases NMR spectroscopy.

Vitamin D and its isomer pre-vitamin D are known to interconvert thermally by a sigmatropic 1,7-hydrogen shift. In vivo, pre-vitamin D has been shown to be an immediate precursor to vitamin D, and both species are found in equilibrium concentrations at physiological temperatures, although that equilibrium appears to be largely in favor of vitamin D. Because both vitamin D and pre-vitamin D are considered to serve the same physiological function, vitamin D assays when reported conventionally comprise the sum of both species. This is consistent with both USP and Ph.Eur. monographs for vitamin D₃ containing products, for example. Available stability data indicate that none of the other isomers will approach the ICH qualification threshold of 1.0% by weight at 24 months, stored at 25° C./60% RH in appropriate packages.

The most prominent degradation products appear to be vitamin D₃ esters formed by transesterification reactions of vitamin D₃ with the medium chain triglycerides (MCT) in the vitamin D₃ granules used in the compositions of the present invention. Structures for some of these vitamin D₃ ester adducts are also shown in FIG. 5. The predominant species correspond to n-octanoate (C₈) and n-decanoate (C₁₀) esters of vitamin D₃. The pre-vitamin D₃ ester adducts can be generated either by reaction of pre-vitamin D₃ with the triglycerides in the vitamin D₃ compound or by thermal conversion from the vitamin D₃ esters. Of the quantifiable degradation products, only the C₈ and C₁₀ vitamin D₃ ester adducts appear to increase to any appreciable extent during the stability study.

Available stability data indicate that these species should not approach the ICH qualification threshold of 1.0% weight at 24 months, stored at less than about 30° C. and at less than about 30% relative humidity (RH) and they are not expected to give rise to safety concerns in any event in embodiments of compositions and methods of the present invention. Studies further show that after 24 months when stored at less than about 30° C. and less than about 30% RH, total degradants of the compositions of the present invention is less than about 5%.

It is also understood that vitamin D can undergo autoxidation through induction by a free radical initiator or spontaneously in solid or solution phase to form a variety of products, some of which have been identified. Representative characterization of vitamin D degradation (and, specifically, vitamin D₃ in this instance) in embodiments of compositions of the present invention confirms the autoxidative lability of vitamin D₃ in which vitamin D₃ was converted to an oil or amorphous solid and exposed to temperatures from 20-40° C. Within hours, HPLC analysis showed extensive destruction of vitamin D₃ and the appearance of many unresolved degradation products exhibiting very low ultraviolet (UV) absorption. At longer exposure times, these absorptions continued to decrease as further reaction occurred.

A more detailed analysis of the autoxidation of vitamin D₃ was carried out using the free radical initiator, azo-bis-isobutyronitrile (AIBN). In this experiment, AIBN was used to initiate the autoxidation of vitamin D₃ in solution. The resulting product profile was characterized by HPLC using UV, mass spectrometric (MS) and evaporative light scattering (ELS) detection. The results showed that: (a) solution-phase autoxidation also can lead to multiple degradation products, (b) autoxidation can lead to gradual destruction of the UV chromophore resulting in an apparent material loss, while ELS detection, on the other hand, afforded significantly better mass recovery, and (c) the mass spectrometric m/z ratios and in some cases the observed UV/vis spectra confirmed the oxidative nature of these reaction products.

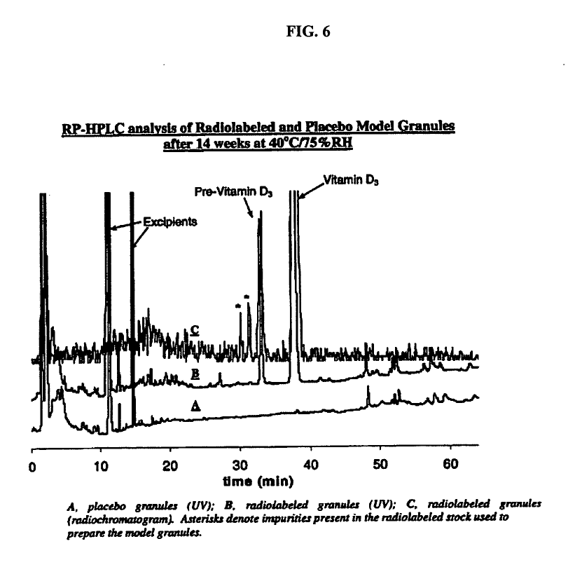

A radiolabel study was conducted in an attempt to characterize vitamin D₃ degradation in a granule formulation used in embodiments of compositions of the present invention comprising about 70 mg alendronate and about 2,800 IU (70 μg) vitamin D₃. Tritium labeled vitamin D₃ was utilized as a means of tracking degraded vitamin D₃, independent of changes in UV absorption characteristics. The radiolabeled vitamin D₃ was incorporated into a formulation that modeled the vitamin D granules used in embodiments of compositions of the present invention, and was then analyzed for stability. The antioxidant level in the model formulation was at a reduced level from antioxidant levels considered desirable for commercial formulations, in order to ensure that degradation occurred within a reasonable timeframe. Samples were analyzed after 14 weeks at 40° C./75% RH and 70° C. using liquid scintillation counting (LSC) and reverse-phase high performance liquid chromatography (RP-HPLC) with simultaneous UV and online radiodetection. A vitamin D₃ loss of approximately 40% was observed for samples stored at 40° C./75% RH conditions, whereas the low temperature controls showed good stability. Results of this analysis are shown in the radiochromatograms for the degraded samples of FIG. 6, which show a large region of unresolved degradates, none of which appears to be a major product. These results provide further evidence that, when not properly stabilized, vitamin D₃ degrades oxidatively into multiple products with reduced UV absorption and that these products account for loss of vitamin D₃. Based on these stability analyses, individual autoxidative degradates are not expected to approach levels that are of a safety concern in tablet embodiments of compositions of the present invention.

The present invention also includes methods of measuring the pharmacokinetic parameters in mammals upon the administration of the compositions of the present invention. The pharmacokinetic parameters that may be measured include, for example, total urinary excretion, urinary excretion, area under the serum-concentration-versus-time curve (AUC), steady state maximum plasma concentration (Cₘₐₓ), time of Cₘₐₓ (Tₘₐₓ), and serum concentration median apparent half-life (t_{1/2}) of a tablet, such as, for example, a tablet comprising about 70 mg alendronate and about 2,800 IU cholecalciferol. These measurements confirm that embodiments of the compositions and methods of the present invention produce pharmaceutically effective levels of alendronate and cholecalciferol in the body (the latter as demonstrated by comparison to recommended daily amounts of a vitamin D compound in the compositions and methods of the present invention).

In an embodiment, the present invention includes methods of measuring cholecalciferol in human serum after administration of a pharmaceutical composition comprising alendronate and cholecalciferol, the method comprising: (1) administering to a human a composition comprising alendronate and cholecalciferol; (2) obtaining from the human a plasma sample; (3) extracting the cholecalciferol from the plasma sample to form a first solution; (4) reacting the cholecalciferol in the first solution with a dienophile to form one or more diels-alder addition products of cholecalciferol; (5) separating the diels-alder addition products of cholecalciferol using high performance liquid chromatography (HPLC) separation; and (6) detecting an amount of cholecalciferol in the sample using mass spectroscopy. In an embodiment of this method, the dienophile comprises 4-phenyl-1,2,4-triazoline-3,5-dione (P-TADO or PTAD). Also, the detecting step may be conducted in a positive ionization mode using a heated nebulizer probe, and may further comprise adding a deuterated internal standard cholecalciferol to each human plasma sample, and extracting, reacting, separating, and detecting the deuterated internal standard cholecalciferol along with the sample cholecalciferol. This method has a limit of quantitation (LOQ) of cholecalciferol of less than about 0.5 ng/mL cholecalciferol when 1 mL of plasma is measured. An embodiment of the present invention is a vitamin D/bisphosphonate composition wherein a plot of serum concentration of a mammal over 120 hours after administration of the composition yields at least one of the following: a least-squares (LS) mean AUC_{(0-120 hr)} of cholecalciferol of about 296.4 ng.h/mL, wherein the pharmacokinetic parameters have been measured without taking into account baseline cholecalciferol serum concentrations; a least-squares (LS) mean AUC_{(0-120 hr)} of about 297.5 ng.h/mL, wherein the pharmacokinetic parameters have been measured by taking into account baseline cholecalciferol serum concentrations using a predose 0 hr serum cholecalciferol concentration as a covariate; and a least-squares (LS) mean AUC_{(0-120 hr)} of about 143.1 ng.h/mL, wherein the pharmacokinetic parameters have been measured by taking into account baseline cholecalciferol serum concentrations using a subtraction of estimated baseline cholecalciferol over the 120 hour period. In another embodiment, the composition comprises a bisphosphonate and cholecalciferol wherein a plot of plasma concentration a mammal over 120 hours after administration of the composition yields at least one of the following: a least-squares (LS) mean for steady state maximum plasma concentration (Cₘₐₓ) of over 120 hours of about 5.9 ng/mL, wherein the pharmacokinetic parameters have been measured without taking into account baseline cholecalciferol serum concentrations; a least-squares (LS) mean for steady state maximum plasma concentration (Cₘₐₓ) of over 120 hours of about 5.9 ng/mL, wherein the pharmacokinetic parameters have been measured by taking into account baseline cholecalciferol serum concentrations using a predose 0 hr serum cholecalciferol concentration as a covariate; and a least-squares (LS) mean for steady state maximum plasma concentration (Cₘₐₓ) of about 4.0 ng/mL, wherein the pharmacokinetic parameters have been measured by taking into account baseline cholecalciferol serum concentrations using a subtraction of estimated baseline cholecalciferol over the 120 hour period.

The present invention also encompasses a composition wherein a plot of the plasma concentration of cholecalciferol of a mammal over 120 hours after administration of the composition yields: a steady state maximum plasma concentration (Cₘₐₓ) of cholecalciferol at an arithmetic mean time of occurrence of Cₘₐₓ (Tₘₐₓ) of about 12 hours, and wherein the pharmacokinetic parameters have been without taking into account baseline cholecalciferol serum concentrations. In a further embodiment, the composition has a plasma concentration median apparent half-life (t_{1/2}) of the cholecalciferol of the composition in mammals that is about 23.8 hours, and the pharmacokinetic parameters have been measured by taking into account baseline cholecalciferol serum concentrations using a subtraction of estimated baseline cholecalciferol procedure.

In order to determine the pharmacokinetic characteristics of the compositions of the present invention, studies of samples from an open-label, randomized, 2-part, 2-period crossover study in 236 healthy non-pregnant women and men age 18 to 65 were conducted. In this study, described in detail in Example 7 below, the pharmacokinetic parameters (AUC_{0-120 hr}, Cₘₐₓ, Tₘₐₓ, and serum concentration median apparent half-life (t_{1/2})) of vitamin D₃ administered as a 70-mg alendronate/2800 IU vitamin D₃ combination tablet relative to a 2800 IU vitamin D₃ tablet were studied. In addition, the urinary excretion of alendronate was studied in the combination tablet in relation to the once-weekly 70 mg tablet of FOSAMAX®. In summary, (1) a 70 mg alendronate/2800 IU vitamin D₃ combination tablet according to the present invention was shown to be bioequivalent to a 70 mg alendronate tablet with respect to alendronate bioavailability; (2) the bioavailability of vitamin D₃ in the 70 mg alendronate/2800 IU vitamin D₃ combination tablet and in a tablet containing 2800 IU vitamin D₃ (without alendronate) was shown to be similar, and (3) a 70 mg alendronate/2800 IU vitamin D₃ combination tablet according to the present invention was shown to be generally well tolerated. Accordingly, it is expected, for example, that once-weekly dosing with a bisphosphonate/vitamin D compound of the present invention will provide vitamin D₃ blood levels and/or therapeutic effects comparable to the vitamin D blood levels and/or therapeutic effects from a recommended daily dose of vitamin D, such as 400 IU vitamin D daily, over the same period as the once-weekly dosing of the bisphosphonate/vitamin D compound.

These and other embodiments of the present invention are further explained in the non-limiting examples that follow.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Example 1

Bisphosphonate and Vitamin D Tablets

A finished drug product is a combination tablet containing alendronate sodium (about 70 mg anhydrous free acid equivalent) and vitamin D₃ (about 2800 I.U. (about 70 μg)), with ingredients identified in Table 1-1. All of the excipients are compendial and were selected to achieve maximum physical and chemical stability.

TABLE 1-1
Tablet Composition

Alendronate Sodium 70 mg/
Vitamin D₃ 2800 I.U.
Tablets

| Ingredient | mg/Tab | Weight % |
|---|---|---|
| Alendronate Sodium | 91.37 | 28.1% |
| Dry Vitamin D₃ 100 | 26.67 | 8.2% |
| granules | | |
| Microcrystalline | 131.0 | 40.3% |
| Cellulose NF | | |
| Lactose Anhydrous | 62.35 | 19.2% |
| NF | | |
| Croscarmellose | 9.740 | 3.0% |
| Sodium NF | | |
| Colloidal Silicon | 0.8120 | 0.25% |
| Dioxide NF | | |
| Magnesium Stearate | 3.0870 | 0.95% |
| NF | | |
| Total | 325 | 100% |

The resulting tablets are used in accordance with the methods of the present invention for preventing, inhibiting, reducing or treating osteoporosis, for example. Similarly, tablets comprising other relative weights of alendronate, on an alendronic acid active basis are prepared including, but not limited to, about 2.5 mg, 5 mg, 8.75 mg, 17.5 mg, 70 mg, 140 mg, 280 mg, 560 mg, or 1120 mg per tablet. Similarly, tablets comprising other relative weights of vitamin D₃ per unit dosage are prepared including, but not limited to, about 1,400, 2,800, 5,600, 7,000 IU, 8,400 IU, 14,000 IU, 28,000, or 36,000 IU per tablet. Such tablets may be administered at intervals ranging from once-weekly to bi-monthly.

### Example 2

Bisphosphonate and Vitamin D Composition

A composition comprising a bisphosphonate and vitamin D may be prepared using mixing and formulation techniques as described in this specification. A composition containing about 35 mg of alendronate, on an alendronic acid active basis, and about 5,600 IU of vitamin D₃ may be prepared using the following relative weights of ingredients.

| Ingredient | Per Tablet | |
|---|---|---|
| Alendronate Monosodium Trihydrate | 45.68 | mg |
| Dry Vitamin D₃ 100 granules | 56 | mg* |
| Anhydrous Lactose, NF | 71.32 | mg |
| Microcrystalline Cellulose, NF | 80.0 | mg |
| Magnesium Stearate, NF | 1.0 | mg |
| Croscarmellose Sodium, NF | 2.0 | mg |

*Granule contains approximately 100,000 IU per one gram; therefore 56 mg of the granule is equivalent to about 5600 IU.

The resulting dosage forms are used in accordance with the methods of the present invention for preventing, inhibiting, reducing or treating osteoporosis, for example. Similarly, dosage forms comprising other relative weights of alendronate, on an alendronic acid active basis are prepared including, but not limited to, about 2.5 mg, 5 mg, 8.75 mg, 17.5 mg, 70 mg, 140 mg, 280 mg, 560 mg, or 1120 mg per tablet. Similarly, dosage forms comprising other relative weights of vitamin D₃ per unit dosage are prepared including, but not limited to, about 1,400, 2,800, 5,600, 7,000 IU, 8,400 IU, 14,000 IU, 28,000, or 36,000 IU per dosage form. Such dosage forms may be administered at intervals ranging from once-weekly to bi-monthly. These dosage forms may be, for example, tablets or capsules

### Example 3

Alendronate and Vitamin D Tablets

Tablets containing about 70 mg of alendronate, on an alendronic acid active basis, and 2800 IU of vitamin D₃, are prepared using methods disclosed herein, using the following relative weights of ingredients:

TABLE 3-1

| Composition (per tablet): | | |
|---|---|---|
| Alendronate sodium | 91.37 | mg^{†} |
| Silicon Dioxide, Colloidal, CAB-O-SIL P | 0.81 | mg |
| Dry Vitamin D₃ 100 granules^{‡} | 26.67 | mg* |
| Cellulose Microcrystalline NF Avicel PH-102 | 131 | mg |
| Lactose NF Anhydrous | 63.35 | mg |
| Croscarmellose Sodium Compendial | 9.74 | mg |
| Magnesium Stearate NF (Non-Bovine) | 3.09 | mg |

^{†}Equivalent to 70.0 mg free acid
^{‡}Dry Vitamin D₃ 100 granules also contained medium chain triglycerides, gelatin, sucrose, butylated hydroxytoluene, starch and sodium aluminum silicate.
*26.67 grams of the Dry Vitamin D₃ 100 granules contains 105,000 IU/g of vitamin D₃.

The resulting tablets are used in accordance with the methods of the present invention for preventing, inhibiting, reducing or treating osteoporosis, for example. Similarly, tablets comprising other relative weights of alendronate, on an alendronic acid active basis are prepared including, but not limited to, about 2.5 mg, 5 mg, 8.75 mg, 17.5 mg, 70 mg, 140 mg, 280 mg, 560 mg, or 1120 mg per tablet. Similarly, tablets comprising other relative weights of vitamin D₃ per unit dosage are prepared including, but not limited to, about 1,400, 2,800, 5,600, 7,000 IU, 8,400 IU, 14,000 IU, 28,000, or 36,000 IU per tablet. Such tablets may be administered at intervals ranging from once-weekly to bi-monthly.

### Example 4

Effect of Vitamin D₃ (Powder Form) on Alendronate Absoprtion

To examine the interaction of vitamin D₃ (cholecalciferol) in powder form on alendronate when administered in a single dosage, a two-period, crossover, study in 14 healthy, nonpregnant women and men, aged 18 to 85 was conducted. Subjects received one alendronate 70-mg tablet in each period. A single dose of vitamin D₃ 5600 IU was coadministered with the alendronate tablet in one of the two periods, based on a computer-generated patient allocation schedule. When vitamin D₃ was administered with alendronate, the vitamin D₃ powder was reconstituted in 60 mL of plain tap water and administered to the subject with the alendronate tablet (Treatment A). The vitamin D₃ bottle was rinsed and filled with 60 mL of plain tap water 3 times, each then administered to the subject. Therefore, a total volume of 240 mL of plain tap water was administered with the vitamin D₃. When alendronate was administered alone, a 240-mL volume of plain tap water was administered with the dose (Treatment B). At least a 14-day washout separated each period. The treatment schematic and allocation are in Table 4-1.

TABLE 4-1
Treatment Schematic and Allocation

| Group | Period 1 | Period 2 |
|---|---|---|
| 1 (N = 7) | A | B |
| ANs 0002, 0004, 0005, 0008, 0009, 0011, 0013 | | |
| 2 (N = 7) | B | A |
| ANs 0001, 0003, 0006, 0007, 0010, 0012, 0014 | | |

Treatment A = 70-mg alendronate tablet with vitamin D₃ 5600 IU.
Treatment B = 70 mg alendronate.

Subjects were sequestered in the study unit the evening prior to each treatment. Following an overnight fast (except water), subjects were administered the respective treatment. Subjects continued to fast following drug administration until a defined meal was administered 2 hours postdose.

Clinical supply information is in Table 4-2. The composition and analytical results for the alendronate tablet and vitamin D₃ used in this study are listed in Tables 4-3 and 4-4.

TABLE 4-2
Clinical Supplies

| Drug | Potency | Dosage Form |
|---|---|---|
| Alendronate | 70 mg | Tablet |
| Vitamin D₃ | 5600 IU | Granules |

TABLE 4-3
Alendronate Tablet Formulation Characteristics

| Composition (per tablet): | | |
|---|---|---|
| Alendronate sodium | 91.37 | mg^{†} |
| Lactose NF Anhydrous | 113.38 | mg |
| Cellulose Microcrystalline NF Avicel 102 | 140.00 | mg |
| Magnesium Stearate Impalpable Powder NF | 1.75 | mg |
| Croscarmellose Sodium NF Type A | 3.50 | mg |

^{†}Equivalent to 70.0 mg free acid.

TABLE 4-4
Vitamin D₃ Granular Powder
Formulation Characteristics

| Composition (per bottle): | |
|---|---|
| Dry vitamin D₃ Type 100 CWS/HP | 51.96 mg^{†} |

^{†}Equivalent to 5600 IU.

All doses were administered following an overnight fast (except for water). Subjects were administered a single alendronate 70-mg tablet with 240 mL of plain tap water. When subjects were administered the vitamin D₃ dose of 5600 IU with alendronate, the subjects were instructed to resuspend and co-administer the alendronate 70-mg tablet with the vitamin D₃ dose (supplied in granulated form and reconstituted in water at the study site). The total volume of liquid administered with each alendronate dose was 240 mL. Subjects remained in the fasted state for an additional 2 hours following study drug administration and subsequently served a defined meal. Subjects remained upright for the 2 hours between drug administration and the defined meal. Each dose period was separated by an interval of at least 14 days.

Urine specimens for alendronate assay were collected for pharmacokinetic analyses over the following intervals: −2 to 0 hour predose, 0 to 8 hours postdose, 8 to 24 hours postdose, and 24 to 36 hours postdose. The urine collection obtained over the 2-hour period just prior to study drug administration provided a baseline alendronate determination. All urine specimens were collected in preweighed polypropylene containers. For the 0- to 8-, 8- to 24-, and 24- to 36-hour postdose urine collections, 12.5 grams of boric acid were added to the containers as a preservative at the beginning of the timed interval. At the end of each timed collection interval, the entire urine collection was weighed, the specific gravity measured, and the net volume determined. The urine specimen was acidified in situ. Five mL of 6.0 N Hydrochloride Acid (HCl) were added per 200 mL of urine to bring the urine specimen pH to ≦2.0. Following acidification, the urine specimen was agitated and a sample was aliquoted into a polypropylene container to be stored frozen (−20° C.) until high-performance liquid chromatography (HPLC) assay was completed. The total urine volume for each period, including the volume of the boric acid and HCl, was used to determine total urinary excretion of alendronate for a given interval.

The analytical method for the determination of alendronate in human urine involved 3 distinct operations: (1) isolation of the analyte and an internal standard (pamidronate) from urine, (2) formation of strongly fluorescent derivatives, and (3) HPLC separation and fluorescence detection of the resulting derivatives. Alendronate and the internal standard were co-precipitated from urine with naturally present phosphates by the addition of calcium chloride and sodium hydroxide. The pellet, isolated by centrifugation, was reconstituted in 1 M hydrochloric acid and applied to an anion-exchange diethylamine (DEA) cartridge in acetate-buffered solution at pH 4. Alendronate was eluted from the DEA cartridge by a solution of 0.20 M sodium citrate and 0.20 M sodium phosphate dibasic (adjusted to pH 9). Alendronate was derived with 2,3-naphthalenedicarboxyaldehyde in the presence of N-acetyl-D-penicillamine at room temperature. The derivative was then applied to a non-silica-based polymeric column composed of the copolymer of styrene and divinyl benzene. The mobile phase was initially composed of 85% 0.025 M sodium citrate, 0.025 M sodium phosphate dibasic (pH 6.95), and 15% acetonitrile at a flow rate of 1 mL/min. Later-eluting endogenous components of urine were removed by increasing acetonitrile to 50%. The assay was validated at between 5 ng/mL and 125 ng/mL, in human urine, with coefficients of variation below 10%. A 5-mL urine sample was required to obtain the 1-ng/mL limit of detection.

The total urinary excretion of alendronate for a given interval (−2 to 0, 0 to 8, 8 to 24, 24 to 36 hours) was determined by multiplying the concentration of alendronate in the analyzed aliquot by the total urine volume (including boric acid and hydrochloric acid) for the interval.

A comparison of the total urinary excretion for the 70-mg alendronate tablet plus 5600 IU vitamin D₃ and the 70-mg alendronate tablet alone was performed using an analysis of variance (ANOVA) model suitable for a 2-period, crossover design. The ANOVA model contained factors for sequence, subject (sequence), period, and treatment. Total urinary excretion was log-transformed. Results from the Shapiro-Wilk test for normality, along with plots of residuals from the model, did not suggest any departure from the assumptions of the ANOVA model. To estimate the relative bioavailability of the 70-mg alendronate tablet plus vitamin D₃ versus the 70-mg alendronate tablet alone, a 95% CI was computed, based upon the t-distribution, for the GMR for total urinary excretion. Additionally, the posterior probability that the true GMR is above the clinically important bound of 0.50 was also calculated.

One subject was dropped from the above analysis since this particular subject had urinary alendronate concentrations for all 3 collection intervals (0 to 8, 8 to 24, and 24 to 36 hours) below limit of quantification for both treatments. Due to a slight imbalance in the ordering of the treatment sequences, the least-squares means for the total urinary excretion are reported. The data from that subject was excluded from analysis.

The least-square means were obtained by back-transformation from the ANOVA model. All p-values were rounded to 3 decimal places prior to reporting. Results for which p≦0.050 is reported are considered statistically significant.

Table 4-5 displays the total urinary excretion of alendronate as a 70-mg alendronate tablet plus vitamin D₃ and the 70-mg alendronate tablet for each subject. Summary statistics along with the GMR, with its corresponding 95% CI, for total urinary excretion of alendronate are in Table 4-6.

The least-squares geometric mean for total urinary excretion was 183.61 for the 70-mg alendronate tablet plus 5600 IU vitamin D₃ and 157.97 μg for the 70-mg alendronate tablet alone. The GMR and its corresponding 95% CI for 70-mg alendronate+vitamin D₃ relative to alendronate alone were 1.16 (0.74, 1.83). The posterior probability that the GMR might be above the clinically important bound of 0.50 was 0.999.

TABLE 4-5
Individual Total Urinary Excretion (μg) of Alendronate Over 36 Hours
Following Single-Dose Administration of 70-mg Alendronate Tablet
Plus 5600 IU Vitamin D₃ and 70 mg Alendronate Administered Alone

| | 70 mg Alendronate Plus | |
|---|---|---|
| | 5600 IU Vitamin D₃ | 70 mg Alendronate |
| | 194.22 | 68.02 |
| | 311.32 | 111.11 |
| | 367.40 | 290.62 |
| | 291.47 | 310.48 |
| | 181.42 | 113.40 |
| | 127.41 | 68.64 |
| | 494.92 | 169.86 |
| | <LOQ^{†} | <LOQ^{†} |
| | 21.75 | 61.29 |
| | 185.46 | 257.75 |
| | 101.51 | 68.73 |
| | 97.86 | 259.69 |
| | 248.71 | 341.90 |
| | 464.51 | 519.58 |
| Arithmetic Mean | 237.54 | 203.16 |
| Standard Deviation | 143.63 | 140.51 |

^{†}<LOQ = Below limit of quantitation of 1 ng/mL.

TABLE 4-6
Summary Statistics and Geometric Mean Ratio With Corresponding 95% CI
of Total Urinary Excretion (μg) of Alendronate Over 36 Hours Alendronate
Following Single-Dose Administration of 70 mg Plus Vitamin D₃
and 70 mg Alendronate Alone

| | | LS^{†} | | | | | | 95% CI^{\|\|} for |
|---|---|---|---|---|---|---|---|---|
| Treatment | N | Mean | Median | Min | Max | SD^{‡} | GMR^{§} | GMR |
| Alendronate + vitamin | 13 | 183.61 | 194.22 | 21.75 | 494.92 | 260.40 | 1.16 | (0.74, 1.83) |
| D₃ | | | | | | | | |
| Alendronate | 13 | 157.97 | 169.86 | 61.29 | 519.58 | 177.07 | | |

Root Mean Squared Error (RMSE) in log scale from ANOVA Model = 0.522 (ln μg).
^{†}LS mean = Least-Squares mean (back-transformed from the log scale).
^{‡}SD = Back-transformed Between-Subject Standard Deviation.
^{§}GMR = Least-Squares mean ratio (alendronate + vitamin D₃/alendronate).
^{||}CI = Confidence Interval.

### Example 5

Effect of Vitamin D₃ (Contained in an Alendronate/Vitamin D Tablet) on Alendronate Absorption

To examine the potential for an interaction between alendronate and orally-administered vitamin D₃, fourteen healthy adult subjects (6 men, 8 women, ages 33-61 yr.) were administered single 70-mg tablets of alendronate, without vitamin D₃, and together with a powdered dose of vitamin D₃, (5600 IU) suspended in 240 mL of water. This study was of an open, randomized, crossover two-way design. The purpose of the study was to obtain a preliminary estimate of the relative bioavailability of alendronate following a 70-mg tablet administered with vitamin D₃, relative to alendronate administered without vitamin D₃.

Alendronate was administered orally as a 70-mg tablet in each of the two periods. In one period, the tablet was administered with vitamin D₃ powder reconstituted in plain tap water and in the alternate period the tablet was taken alone with plain tap water. Urine was collected for two hours preceding and 36 hours following each dose of alendronate for analytical determination of excreted alendronate. Relative bioavailability was estimated based on total urinary recovery of alendronate over the 36 hours post-dose.

Urinary recovery of alendronate following the dose of 70-mg alendronate without vitamin D₃ was 202 μg with a 90% CI of (126 μg, 279 μg), recoveries following the 70-mg dose administered together with vitamin D₃, averaged 238 μg with a 90% CI of (59 μg, 316 μg). The geometric mean ratio (90% CI) was estimated at 1.18 (0.80, 1.74). This investigation shows that oral administration of vitamin D₃ together with an oral dose of alendronate has minimal to no effect on the bioavailability of alendronate.

### Example 6

Stability Study of Vitamin D₃ and Alendronate

The stability of a composition of the invention in the form of a combination tablet containing alendronate sodium (70 mg anhydrous free acid equivalent) and vitamin D₃ (2800 I.U./70 μg) has been studied. Table 6-1 contains a the tablet composition of an embodiment of an alendronate/vitamin D combination tablet. All of the excipients are compendial grade and were selected to achieve maximum physical and chemical stability.

TABLE 6-1
Tablet Composition

Alendronate Sodium 70 mg/
Vitamin D₃ 2800 I.U. Tablets

| Ingredient | mg/Tab | Weight % |
|---|---|---|
| Alendronate Sodium | 91.37 | 28.1% |
| Dry Vitamin D₃ 100 granules | 26.67* | 8.2% |
| Microcrystalline Cellulose NF | 131.0 | 40.3% |
| Lactose Anhydrous NF | 62.35 | 19.2% |
| Croscarmellose Sodium NF | 9.740 | 3.0% |
| Colloidal Silicon Dioxide NF | 0.8120 | 0.25% |
| Magnesium Stearate NF | 2.275 | 0.7% |
| (Intragranular) | | |
| Magnesium Stearate NF | 0.8120 | 0.25% |
| (Extragranular) | | |
| Total | 325 | 100% |

*26.67 grams of the Dry Vitamin D₃ 100 granules contains 105,000 IU/g of vitamin D₃

The alendronate assay and dissolution methods may employ reversed-phase HPLC with pre-column 9-fluorenylmethyl chloroformate (FMOC) derivatization, similar to methods already reported for FOSAMAX® tablets. The vitamin D₃ assay and degradates method may also be a reversed-phase, gradient HPLC method (RP-HPLC) capable of resolving and quantitating vitamin D₃ and multiple potential degradation products of vitamin D₃. The vitamin D₃ content uniformity and dissolution assays also employ reversed-phase HPLC. The dissolution method may use a surfactant medium (1% SDS) due to the poor aqueous solubility of vitamin D₃. Due to the low vitamin D₃ potency (70 μg) of the combination tablet, one may use three tablets to 500 mL of medium to obtain a suitable signal.

Fifty-two weeks of assay and degradate data are provided below for a batch of the combination tablets stored at 30° C./65% RH and 40° C./75% RH (See Tables 6-2 and 6-3). These data demonstrate the acceptable stability of an embodiment of a compositions of the present invention, although the data generated do indicate that there is slight degradation of vitamin D₃. Greater degradation is found at higher temperatures in the aluminum blisters and the HDPE bottles without desiccant.

TABLE 6-2
Summary of Vitamin D₃ Stability Assay Results:
Alendronate Sodium 70 mg/Vitamin D₃ 2800 I.U. Combination Tablets

| | | Vitamin D₃ (% Label | | | | | |
|---|---|---|---|---|---|---|---|
| | | Claim) 75 ml HDPE bottle | | | | | |
| | | w/foil induction seal, | | | Vitamin D₃ (% Label | | |
| | | 1 g desiccant, | | | Claim) Foil to Foil | | |
| | | 4 tablets per bottle | | | Aluminum Blister | | |
| Storage Condition | Weeks^{†} | Lot 001 | Lot 002 | Lot 003 | Lot 001 | Lot 002 | Lot 003 |
| Initial | 0 | 98.6 | 97.3 | 99.2 | 98.6 | 97.3 | 99.2 |
| 25° C./60% RH | 13 | NT | 97.3 | 99.5 | 99.2 | 97.7 | NT |
| | 26 | 99.9 | 98.0 | NT | 100.8 | NT | 99.6 |
| | 39 | 99.0 | NT | 98.1 | NT | 93.8 | 97.1 |
| | 44 | 99.1 | 97.1 | 99.6 | 97.9 | 95.4 | 99.1 |
| | 52 | 98.2 | 96.4 | 99.3 | 98.8 | 94.9 | 99.3 |
| 30° C./65% RH | 13 | 100.1 | 96.8 | NT | 99.3 | NT | 99.1 |
| | 26 | 99.7 | NT | 99.5 | NT | 96.7 | 99.8 |
| | 39 | NT | 94.4 | 97.1 | 97.6 | 94.3 | NT |
| | 44 | 97.3 | 95.7 | 97.6 | 97.9 | 96.1 | 98.0 |
| | 52 | 97.5 | 95.4 | 97.5 | 97.7 | 94.1 | 97.1 |
| 40° C./75% RH | 13 | 99.3 | 96.0 | 99.0 | 97.1 | 96.1 | 97.8 |
| | 26 | 97.1 | 94.5 | 96.9 | 96.8 | 94.7 | 97.6 |

^{†}The theoretical timepoint in weeks is indicated.
NT = Not tested.

TABLE 6-3
Summary of Vitamin D₃ Degradation Stability Results: Alendronate Sodium
70 mg/Vitamin D₃ 2800 I.U. Combination Tablets, Foil to Foil Aluminum Blister

| | | | % Label Claim | | |
|---|---|---|---|---|---|
| | Storage | | (wt % relative to Vitamin D) | | |
| Degradate | Condition | Weeks^{†} | Lot 001 | Lot 002 | Lot 003 |
| 0.74RRT | Initial | 0 | 0.4 | 0.3 | 0.3 |
| (trans-vitamin D₃) | 25° C./60% RH | 13 | 0.2 | 0.3 | NT |
| | | 26 | 0.2 | NT | 0.2 |
| | | 39 | NT | 0.3 | 0.2 |
| | | 44 | 0.2 | 0.3 | 0.2 |
| | | 52 | 0.2 | 0.3 | 0.2 |
| | 30° C./65% RH | 13 | 0.2 | NT | 0.3 |
| | | 26 | NT | 0.3 | 0.2 |
| | | 39 | 0.2 | 0.3 | NT |
| | | 44 | 0.2 | 0.3 | 0.2 |
| | | 52 | 0.2 | 0.3 | 0.2 |
| | 40° C./75% RH | 13 | 0.2 | 0.3 | 0.2 |
| | | 26 | 0.1 | 0.2 | 0.1 |
| 0.78RRT | Initial | 0 | 0.0^{‡} | 0.0^{‡} | 0.0^{‡} |
| (vitamin D₃ isomer) | 25° C./60% RH | 13 | 0.0^{‡} | 0.1 | NT |
| | | 26 | 0.1 | NT | 0.1 |
| | | 39 | NT | 0.1 | 0.1 |
| | | 44 | 0.1 | 0.1 | 0.1 |
| | | 52 | 0.1 | 0.1 | 0.1 |
| | 30° C./65% RH | 13 | 0.1 | NT | 0.1 |
| | | 26 | NT | 0.1 | 0.1 |
| | | 39 | 0.2 | 0.2 | NT |
| | | 44 | 0.2 | 0.2 | 0.2 |
| | | 52 | 0.2 | 0.2 | 0.2 |
| | 40° C./75% RH | 13 | 0.2 | 0.2 | 0.2 |
| | | 26 | 0.3 | 0.3 | 0.3 |
| 0.96RRT | Initial | 0 | 0.2 | 0.3 | 0.2 |
| (vitamin D₃ isomer) | 25° C./60% RH | 13 | 0.2 | 0.2 | NT |
| | | 26 | 0.2 | NT | 0.1 |
| | | 39 | NT | 0.2 | 0.1 |
| | | 44 | 0.1 | 0.2 | 0.1 |
| | | 52 | 0.1 | 0.2 | 0.1 |
| | 30° C./65% RH | 13 | 0.2 | NT | 0.2 |
| | | 26 | NT | 0.2 | 0.1 |
| | | 39 | 0.1 | 0.2 | NT |
| | | 44 | 0.1 | 0.2 | 0.1 |
| | | 52 | 0.1 | 0.2 | 0.1 |
| | 40° C./75% RH | 13 | 0.1 | 0.2 | 0.1 |
| | | 26 | 0.0^{‡} | 0.0^{‡} | 0.0^{‡} |
| 1.09RRT | Initial | 0 | NR | NR | NR |
| (vitamin D₃ degradate) | 25° C./60% RH | 13 | 0.0^{‡} | 0.0^{‡} | NT |
| | | 26 | 0.0^{‡} | NT | 0.0^{‡} |
| | | 39 | NT | 0.0^{‡} | 0.0^{‡} |
| | | 44 | 0.0^{‡} | 0.0^{‡} | 0.0^{‡} |
| | | 52 | 0.0^{‡} | 0.0^{‡} | 0.0^{‡} |
| | 30° C./65% RH | 13 | 0.0^{‡} | NT | 0.0^{‡} |
| | | 26 | NT | 0.0^{‡} | 0.0^{‡} |
| | | 39 | 0.0^{‡} | 0.0^{‡} | NT |
| | | 44 | 0.0^{‡} | 0.1 | 0.0^{‡} |
| | | 52 | 0.1 | 0.1 | 0.1 |
| | 40° C./75% RH | 13 | 0.0^{‡} | 0.0^{‡} | 0.0^{‡} |
| | | 26 | 0.2 | 0.2 | 0.1 |
| 1.39RRT | Initial | 0 | 0.1 | 0.1 | 0.1 |
| (C8 vitamin D₃ ester) | 25° C./60% RH | 13 | 0.2 | 0.1 | NT |
| | | 26 | 0.2 | NT | 0.2 |
| | | 39 | NT | 0.2 | 0.2 |
| | | 44 | 0.3 | 0.2 | 0.3 |
| | | 52 | 0.3 | 0.3 | 0.3 |
| | 30° C./65% RH | 13 | 0.2 | NT | 0.2 |
| | | 26 | NT | 0.2 | 0.3 |
| | | 39 | 0.4 | 0.3 | NT |
| | | 44 | 0.4 | 0.4 | 0.4 |
| | | 52 | 0.5 | 0.4 | 0.5 |
| | 40° C./75% RH | 13 | 0.3 | 0.3 | 0.3 |
| | | 26 | 0.6 | 0.5 | 0.6 |
| 1.52RRT | Initial | 0 | 0.0^{‡} | 0.0^{‡} | 0.0^{‡} |
| (C10 vitamin D₃ ester) | 25° C./60% RH | 13 | 0.0^{‡} | 0.0^{‡} | NT |
| | | 26 | 0.1 | NT | 0.1 |
| | | 39 | NT | 0.1 | 0.1 |
| | | 44 | 0.2 | 0.1 | 0.2 |
| | | 52 | 0.2 | 0.2 | 0.2 |
| | 30° C./65% RH | 13 | 0.0^{‡} | NT | 0.1 |
| | | 26 | NT | 0.1 | 0.2 |
| | | 39 | 0.2 | 0.2 | NT |
| | | 44 | 0.2 | 0.2 | 0.2 |
| | | 52 | 0.3 | 0.2 | 0.3 |
| | 40° C./75% RH | 13 | 0.2 | 0.2 | 0.2 |
| | | 26 | 0.4 | 0.3 | 0.4 |
| Total degradates | Initial | 0 | 0.7 | 0.7 | 0.6 |
| | 25° C./60% RH | 13 | 0.6 | 0.8 | NT |
| | | 26 | 0.8 | NT | 0.8 |
| | | 39 | NT | 1.0 | 0.9 |
| | | 44 | 0.9 | 1.0 | 0.9 |
| | | 52 | 1.0 | 1.1 | 1.0 |
| | 30° C./65% RH | 13 | 0.7 | NT | 0.8 |
| | | 26 | NT | 1.0 | 0.9 |
| | | 39 | 1.0 | 1.1 | NT |
| | | 44 | 1.1 | 1.3 | 1.1 |
| | | 52 | 1.3 | 1.4 | 1.4 |
| | 40° C./75% RH | 13 | 0.9 | 1.1 | 0.9 |
| | | 26 | 1.6 | 1.6 | 1.5 |

^{†}The theoretical timepoint in weeks is indicated.
^{‡}0.0 represents results <0.1% or Not Detected.
NT = Not tested.
NR = Not reported.

Summary of Vitamin D₃ Degradation Stability Results: Alendronate Sodium
70 mg/Vitamin D₃ 2800 I.U. Combination Tablets, 75 cc HDPE Bottle,
Tablets per Bottle, and One 1-gram Desiccant

| | | | % Label Claim | | |
|---|---|---|---|---|---|
| | Storage | | (wt % relative to Vitamin D) | | |
| Degradate | Condition | Weeks^{†} | Lot 001 | Lot 002 | Lot 003 |
| 0.74RRT | Initial | 0 | 0.4 | 0.3 | 0.3 |
| (trans-vitamin D₃) | 25° C./60% RH | 13 | NT | 0.4 | 0.3 |
| | | 26 | 0.2 | 0.3 | NT |
| | | 39 | 0.2 | NT | 0.2 |
| | | 44 | 0.2 | 0.3 | 0.2 |
| | | 52 | 0.2 | 0.3 | 0.2 |
| | 30° C./65% RH | 13 | 0.2 | 0.3 | NT |
| | | 26 | 0.2 | NT | 0.2 |
| | | 39 | NT | 0.3 | 0.2 |
| | | 44 | 0.2 | 0.3 | 0.2 |
| | | 52 | 0.2 | 0.3 | 0.2 |
| | 40° C./75% RH | 13 | 0.2 | 0.3 | 0.2 |
| | | 26 | 0.2 | 0.2 | 0.2 |
| 0.78RRT | Initial | 0 | 0.1 | 0.0^{‡} | 0.0^{‡} |
| (vitamin D₃ isomer) | 25° C./60% RH | 13 | NT | 0.0^{‡} | 0.0^{‡} |
| | | 26 | 0.1 | 0.1 | NT |
| | | 39 | 0.1 | NT | 0.1 |
| | | 44 | 0.1 | 0.1 | 0.1 |
| | | 52 | 0.1 | 0.1 | 0.1 |
| | 30° C./65% RH | 13 | 0.1 | 0.1 | NT |
| | | 26 | 0.1 | NT | 0.1 |
| | | 39 | NT | 0.1 | 0.1 |
| | | 44 | 0.2 | 0.1 | 0.1 |
| | | 52 | 0.2 | 0.2 | 0.2 |
| | 40° C./75% RH | 13 | 0.1 | 0.1 | 0.1 |
| | | 26 | 0.2 | 0.2 | 0.2 |
| 0.96RRT | Initial | 0 | 0.2 | 0.3 | 0.2 |
| (vitamin D₃ isomer) | 25° C./60% RH | 13 | NT | 0.2 | 0.2 |
| | | 26 | 0.1 | 0.2 | NT |
| | | 39 | 0.1 | NT | 0.1 |
| | | 44 | 0.1 | 0.2 | 0.1 |
| | | 52 | 0.1 | 0.2 | 0.1 |
| | 30° C./65% RH | 13 | 0.2 | 0.2 | NT |
| | | 26 | 0.1 | NT | 0.1 |
| | | 39 | NT | 0.2 | 0.1 |
| | | 44 | 0.1 | 0.2 | 0.1 |
| | | 52 | 0.1 | 0.2 | 0.1 |
| | 40° C./75% RH | 13 | 0.2 | 0.2 | 0.1 |
| | | 26 | 0.0^{‡} | 0.1 | 0.0^{‡} |
| 1.09RRT | Initial | 0 | NR | NR | NR |
| (vitamin D₃ degradate) | 25° C./60% RH | 13 | NT | NR | NR |
| | | 26 | 0.0^{‡} | 0.0^{‡} | 0.0^{‡} |
| | | 39 | 0.0^{‡} | NT | 0.0^{‡} |
| | | 44 | 0.1 | 0.0^{‡} | 0.0^{‡} |
| | | 52 | 0.1 | 0.0^{‡} | 0.1 |
| | 30° C./65% RH | 13 | 0.0^{‡} | 0.0^{‡} | NT |
| | | 26 | 0.1 | NT | 0.0^{‡} |
| | | 39 | NT | 0.1 | 0.1 |
| | | 44 | 0.2 | 0.1 | 0.1 |
| | | 52 | 0.1 | 0.1 | 0.2 |
| | 40° C./75% RH | 13 | 0.1 | 0.1 | NR |
| | | 26 | 0.2 | 0.1 | 0.1 |
| 1.39RRT | Initial | 0 | 0.1 | 0.1 | 0.1 |
| (C8 vitamin D₃ ester) | 25° C./60% RH | 13 | NT | 0.1 | 0.1 |
| | | 26 | 0.2 | 0.2 | NT |
| | | 39 | 0.2 | NT | 0.2 |
| | | 44 | 0.2 | 0.2 | 0.2 |
| | | 52 | 0.2 | 0.2 | 0.3 |
| | 30° C./65% RH | 13 | 0.1 | 0.1 | NT |
| | | 26 | 0.2 | NT | 0.2 |
| | | 39 | NT | 0.3 | 0.3 |
| | | 43 | 0.3 | 0.3 | 0.3 |
| | | 52 | 0.4 | 0.3 | 0.4 |
| | 40° C./75% RH | 13 | 0.2 | 0.2 | 0.2 |
| | | 26 | 0.5 | 0.4 | 0.5 |
| 1.52RRT | Initial | 0 | 0.0^{‡} | 0.0^{‡} | 0.0^{‡} |
| (C10 vitamin D₃ ester) | 25° C./60% RH | 13 | NT | 0.0^{‡} | 0.0^{‡} |
| | | 26 | 0.0^{‡} | 0.0^{‡} | NT |
| | | 39 | 0.1 | NT | 0.1 |
| | | 44 | 0.1 | 0.1 | 0.1 |
| | | 52 | 0.1 | 0.1 | 0.1 |
| | 30° C./65% RH | 13 | 0.0^{‡} | 0.0^{‡} | NT |
| | | 26 | 0.1 | NT | 0.1 |
| | | 39 | NT | 0.1 | 0.2 |
| | | 44 | 0.2 | 0.2 | 0.2 |
| | | 52 | 0.2 | 0.2 | 0.2 |
| | 40° C./75% RH | 13 | 0.1 | 0.1 | 0.1 |
| | | 26 | 0.3 | 0.2 | 0.3 |
| Total degradates | Initial | 0 | 0.7 | 0.7 | 0.6 |
| | 25° C./60% RH | 13 | NT | 0.7 | 0.6 |
| | | 26 | 0.6 | 0.8 | NT |
| | | 39 | 0.8 | NT | 0.8 |
| | | 44 | 1.0 | 0.9 | 0.8 |
| | | 52 | 1.0 | 1.0 | 1.0 |
| | 30° C./65% RH | 13 | 0.7 | 0.8 | NT |
| | | 26 | 0.9 | NT | 0.9 |
| | | 39 | NT | 1.1 | 1.0 |
| | | 44 | 1.2 | 1.2 | 1.1 |
| | | 52 | 1.2 | 1.3 | 1.2 |
| | 40° C./75% RH | 13 | 1.0 | 1.1 | 0.9 |
| | | 26 | 1.2 | 1.3 | 1.2 |

^{†}The theoretical timepoint in weeks is indicated.
^{‡}0.0 represents results <0.1% or Not Detected.
NT = Not tested.
NR = Not reported.

### Example 7

Pharmacokinetics of Vitamin D₃ and Alendronate

An open-label, randomized, two-part, two-period, crossover study was conducted with 236 healthy non pregnant women and men aged 18 to 65. The study was conducted in two parts (Parts I and II) with each consisting of a two-period, crossover design. Each subject participated in one part of the study only (i.e., each subject participated only in Part I or only in Part II). Subjects entered into the study sequentially within each part of the study, with a washout period of at least 12 days between treatments periods within each part of the study. Part I included Treatments A and B, and Part II included Treatments A and C. The Treatments consisted of the following: Treatment A—single dose of a 70 mg alendronate/2800 IU vitamin D₃ combination tablet according to Table 7-3 below; Treatment B—single dose of a 70 mg alendronate tablet according to Table 7-2 below; Treatment C—a single dose of a 2800 IU vitamin D₃ tablet (containing placebo excipients to replace alendronate) according to Table 7-4 below.

In Part I, urine was collected starting 2 hours prior to, and over the 36 hours following, dose administration in each period for determination of total urinary excretion of alendronate. In Part II, blood samples were collected for serum vitamin D₃ determination in each period at −24, −18, −12, and −6 hours predose, at 0 hour (just prior to drug administration), and at selected times over the 120 hours following dose administration.

Part I of the study evaluated the bioequivalence of alendronate in the 70 mg alendronate/2800 IU vitamin D₃ combination tablet according to Table 7-3 below, and a 70-mg alendronate tablet according to Table 7-2 below. Part II of the study evaluated serum pharmacokinetics (AUC_{0-120 hr}, Cₘₐₓ) of vitamin D₃ obtained following administration of the 70 mg alendronate/2800 IU vitamin D₃ combination tablet and the 2800 IU vitamin D₃ tablet. The 2800 IU vitamin D₃ tablet contained 2800 IU vitamin D₃ and the inactive excipients in the alendronate/vitamin D₃ combination tablet.

The primary pharmacokinetic parameter in Part I was total urinary excretion of alendronate from 0 to 36 hours following oral-dose administration. Determination of total urinary excretion of alendronate was consistent with previous studies characterizing the oral bioavailability of alendronate through urinary excretion, since plasma concentrations following oral administration are low and difficult to detect.

The primary pharmacokinetic parameters in Part II were AUC_{0-120 hr} and Cₘₐₓ of vitamin D₃. Blood was collected for determination of serum vitamin D₃ concentration at the following time points in each period: −24, −18, −12, −6 hours pre-dose, 0 hour (immediately prior to drug administration), and 2, 3, 5, 7, 16, 24, 36, 48, 72, 96, and 120 hours post-dose.

Serum vitamin D₃ concentrations for 24 hours prior to study drug administration were collected to provide an indication of the behavior of endogenous levels of vitamin D₃ over 24 hours in a controlled environment. Since vitamin D₃ is synthesized in the skin via exposure to ultraviolet light, subjects were housed in the study unit and not exposed to direct sunlight during the duration of the pharmacokinetic-sampling periods (e.g., for 144 hours, from 24 hours pre-dose until 120 hours post-dose). Subjects were required to wear sunblock (SPF 45) and limit sun exposure throughout the entire study including the washout period. Subjects were also restricted from eating foods known to be high in vitamin D₃ (e.g., salmon, herring, mackerel, cod, tuna fish, swordfish oysters, and sardines) as well as foods known to be supplemented with vitamin D₃ (e.g., certain cereals, fortified milk and some yogurts).

Each subject in Part I received a single oral dose of 70 mg alendronate/2800 IU vitamin D₃ combination tablet and a single oral dose of 70 mg alendronate in a randomized, crossover fashion. Subjects in Part II received a single oral dose of 70 mg alendronate/2800 IU vitamin D₃ combination tablet and a single oral dose of a 2800 IU vitamin D₃ tablet in a randomized crossover fashion. Doses were administered with 240 mL of plain tap water following an overnight fast (except water), beginning at 2100 hours the evening prior to dosing. Subjects were instructed not to lie down and to remain upright (at least at a 45° angle, sitting or standing) between drug administration and the defined meal. Subjects fasted until the standard meal, which was administered at 2 hours post-dose. The procedures for administration of the alendronate/vitamin D₃ combination tablet were the same as those for alendronate.

The compositions administered in the study are as set forth in the tables 7-1 through 7-4 below:

TABLE 7-1
Clinical Supplies

| | | Dosage |
|---|---|---|
| Drug | Potency | Form |
| Alendronate sodium/vitamin D₃ | 70 mg/2800 IU | Tablet |
| Alendronate sodium | 70 mg | Tablet |
| Vitamin D₃ | 2800 IU | Tablet |

TABLE 7-2
70-mg Alendronate Tablet Formulation

| Composition (per tablet): | | |
|---|---|---|
| Alendronate sodium | 91.37 | mg^{‡} |
| Microcrystalline Cellulose NF | 140.0 | mg |
| Lactose Anhydrous NF | 113.4 | mg |
| Croscarmellose Sodium NF | 3.50 | mg |
| Magnesium Stearate NF | 1.75 | mg |
| Content Assay (Alendronic Acid): | | |
| Mean | 70.42 | mg |
| Range | 66.5-73.5 | mg |

^{†}Manufactured by the Merck Manufacturing Division as commercial Alendronate Sodium 70 mg Tablet product.
^{‡}Equivalent to 70.0 mg anhydrous free acid.

TABLE 7-3
70-mg Alendronate/2800 IU Vitamin D₃ Combination Tablet Formulation

| Composition (per tablet): | | |
|---|---|---|
| Alendronate Sodium | 91.37 | mg^{†} |
| Dry Vitamin D₃ 100 granules | 26.67 | mg^{‡} |
| Lactose Anhydrous NF | 62.32 | mg^{§} |
| Microcrystalline Cellulose NF | 131.00 | mg |
| Colloidal Silicon Dioxide NF | 0.812 | mg |
| Croscarmellose Sodium NF | 9.740 | mg |
| Magnesium Stearate NF (Intragranular) | 2.275 | mg |
| Magnesium Stearate NF (Extragranular) | 0.812 | mg |
| Content Assay (Alendronic Acid): | | |
| Mean | 70.5 | mg |
| RSD | 0.89% | |
| Range | 69.7-71.6 | mg |
| Content Assay (Vitamin D₃): | | |
| Mean | 2742 | IU |
| RSD | 1.59% | |
| Range | 2643-2822 | IU |

^{†}Equivalent to 70.0 mg anhydrous free acid
^{‡}Vitamin D₃ Dry Pharm Grade granules also contained medium chain triglycerides, gelatin, sucrose, butylated hydroxytoluene, starch and sodium aluminum silicate. Adjusted based on assay. Quantity specified assumes and assay of 105,000 I.U./g.
^{§}Adjusted based on quantity of Vitamin D₃ 100,000 I.U./g added in order to achieve final target tablet weight of 325 mg.

TABLE 7-4
2800 IU Vitamin D₃ Tablet Formulation

| Composition (per tablet): | | |
|---|---|---|
| Dry Vitamin D₃ 100 granules | 26.72 | mg^{†} |
| Microcrystalline Cellulose NF | 97.68 | mg |
| Lactose Anhydrous NF | 46.61 | mg |
| Croscarmellose Sodium NF | 5.34 | mg |
| Colloidal Silicon Dioxide NF | 0.445 | mg |
| Magnesium Stearate NF | 1.336 | mg |
| Content Assay (Vitamin D₃): | | |
| Mean | 2789 | IU |
| RSD | 2.7% | |
| Range | 2660-2957 | IU |

^{†}Dry Vitamin D₃ 100 granules also contained medium chain triglycerides, gelatin, sucrose, butylated hydroxytoluene, starch and sodium aluminum silicate Adjusted based on assay. Quantity specified assumes an assay of 105,000 I.U./g. Tablet weight varied based on quantity of vitamin D₃ 100,000 I.U./g added.

The area under the serum concentration-versus-time curve from 0 to 120 hours postdose (AUC_{0-120 hr}) was calculated using the unadjusted concentrations of vitamin D₃ (Cₜ) by the trapezoidal method to the last sample collection. Samples with concentrations lower than the assay's limit of quantitation (LOQ) were assigned a value of zero for calculation purposes. Maximum observed concentrations (Cₘₐₓ) and time of Cₘₐₓ (Tₘₐₓ) were obtained by inspection of the measured concentrations of vitamin D₃ in serum and the actual recorded times of sample collection. Concentration profiles of vitamin D₃ in serum were also measured in three different ways, two of which account for baseline vitamin D₃ serum concentrations in the manner discussed in below. AUC_{0-120 hr}, Cₘₐₓ and Tₘₐₓ were calculated in the same manner.

The bioavailability of the 70 mg alendronate tablet/2800 IU vitamin D₃ combination tablet relative to the 70 mg alendronate alone tablet was estimated using the GMR for the total urinary excretion of alendronate from the alendronate/vitamin D₃ combination tablet versus the 70-mg alendronate-alone tablet. The relative bioavailability of the 70 mg alendronate/2800 IU vitamin D₃ combination tablet with respect to 2800-IU vitamin D₃ tablet alone was estimated using the GMR (alendronate plus vitamin D₃/vitamin D₃ alone) for AUC_{0-120 hr} and Cₘₐₓ.

The vitamin D₃ single-dose pharmacokinetics following the administration of 70 mg alendronate/2800 IU vitamin D₃ combination tablet and 2800 IU vitamin D₃ tablet were compared using three different approaches. In the first approach, the vitamin D₃ pharmacokinetics for endogenous vitamin D₃ serum concentrations were compared following the administration of the two treatments.

Using this approach, the vitamin D₃ single-dose pharmacokinetics (AUC_{0-120 hr} and Cₘₐₓ) for endogenous vitamin D₃ serum concentrations following the administration of 70 mg alendronate/2800 IU vitamin D₃ combination tablet and 2800 IU vitamin D₃ tablet were compared using an ANOVA model appropriate for a 2-period, crossover design. Appropriate transformations were used on the pharmacokinetic parameters (i.e., log-transformation for AUC_{0-120 hr}, C_{max,} ranks for Tₘₐₓ, and inverse for apparent t_{1/2}. Back-transformed summary statistics and inferential results were reported. The assumptions of the ANOVA model were tested for normality. The normality assumption was generally satisfied for AUC_{0-120 hr} and Cₘₐₓ.

To estimate the bioavailability of vitamin D₃ in the 70 mg alendronate/2800 IU vitamin D₃ combination tablet relative to that of the 2800 IU vitamin D₃ tablet, a 90% CI, based upon the t-distribution, was calculated for the GMR (70 mg alendronate/2800 IU vitamin D₃ combination tablet/2800 IU vitamin D₃ tablet) of AUC_{0-120 hr} and Cₘₐₓ and then compared to the pre-specified bioequivalence bounds of (0.80, 1.25). Summary statistics and between-treatment comparisons were also provided for the Tₘₐₓ of vitamin D₃.

As another way to measure the pharmacokinetic parameters, consideration of pre-dose vitamin D₃ in the plasma specifically led to the development of a model for the observed changes in vitamin D₃ concentrations during the experimental period. This model allowed for the subtraction of the contributions from the baseline vitamin D₃ serum concentrations and enable the estimation of pharmacokinetic parameters arising exclusively from the oral administration of this compound. The model rested on the following assumptions: (1) background concentrations change in an approximately linear fashion as a function of time (Cₜ=Cᵢ+Cₘ·t, where Cᵢ and Cₘ are the intercept and slope of a straight line and t is the number of hours relative to dose administration) when no exogenous vitamin D₃ is administered; (2) the pharmacokinetic behaviors of endogenous and ingested vitamin D₃ are independent of each other, that is, the body handles the endogenously available vitamin D₃ in the same manner, whether additional doses are ingested or not (treated and untreated in the context of this study), and ingested vitamin D₃ is also handled similarly in the presence of varying amounts of this compound in the body previous to dose administration; (3) return of the concentration profile to baseline following a dose takes place in a pharmacokinetic manner similar to that observed when exogenous compounds (most drug products) are administered, (i.e., the terminal phase of return to baseline will be log linear).

Based on these assumptions, a function describing the sum of a baseline of the form Cₜ=Cᵢ+Cₘ·t and a two-compartment model (See equation below) was fitted to individual Cₜ vs. t profiles (−24 to 120 h post-dose) by a least-squares minimization method using a Generalized Reduced Gradient nonlinear optimization method implemented in Microsoft EXCEL (Solver Routine). The fit was constrained to yield a terminal phase of approach-to-baseline approximating log linear behavior. The best fit coefficients for each profile were then used to interpolate the values of the baseline in the range of 0 to 120 hr post-dose and the interpolated baseline subtracted from each profile.
Cₜ=Cᵢ+Cₘt+Ae^{-k}^{_{d}}^{(t-t}^{_{lag}}⁾+Be^{-k}^{ₑₗ}^{(t-t}^{_{lag}}⁾−(A+B)e^{-k}^{ₐ}^{(t-t}^{_{lag}}⁾
where
   t=time relative to dose administration
   Cₜ=Predicted concentration of vitamin D₃ in serum
   Cᵢ=Predicted value of baseline at t=0
   Cₘ=Slope of predicted baseline
      And A, B, k_{d}, kₑₗ and kₐ are parameters of a two-compartment model with first order absorption and t_{lag} is the individual delay in absorption following oral administration of vitamin D₃.

Pharmacokinetic parameters measured using this method (AUC_{0-120 hr}, Cₘₐₓ, Tₘₐₓ) were calculated in the same manner as described using the first measurement method. Summary statistics for total urinary excretion of alendronate over 36 hours are presented in Table 7-5 below. After, a single dose, the LS means for total urinary excretion of alendronate were 197.5 and 191.9 μg for the 70 mg alendronate/2800 IU vitamin D₃ combination tablet and 70-mg alendronate-alone tablet, respectively. The GMR and corresponding 90% CI for the total urinary excretion of alendronate (70 mg alendronate/2800 IU vitamin D₃ combination tablet versus the 70 mg alendronate-alone tablet) was 1.03 (0.91, 1.17). The 90% CI fell within the pre-specified bioequivalence bounds of (0.80, 1.25).

TABLE 7-5
Summary Statistics and GMR With Corresponding 90% Confidence Intervals
for Alendronate (μg) Total Urinary Excretion Following Single Dose
Administration of 70 mg Alendronate/2800 IU Vitamin D₃
Combination Tablet or 70 mg Alendronate-Alone Tablet

| | | LS^{†} | | | | | | 90% CI^{\|\|} |
|---|---|---|---|---|---|---|---|---|
| Treatment | N | Mean | Median | Min | Max | SD^{‡} | GMR^{§} | for GMR |
| Alendronate + Vit | 207 | 197.5 | 209.8 | 11.3 | 3617.7 | 329.1 | 1.03 | (0.91, |
| D₃ | | | | | | | | 1.17) |
| Alendronate | 207 | 191.9 | 204.4 | 0.1 | 1629.6 | 522.2 | | |

Root Mean Squared Error (RMSE) in log scale from ANOVA Model = 0.778.
^{†}LS = Least-Squares (Back-transformed from the log scale).
^{‡}SD = Between-Subject Standard Deviation back-transformed from log scale.
^{§}GMR = Geometric Mean Ratio (LS Mean of alendronate + vit D₃/LS Mean of alendronate).
^{||}CI = Confidence Interval.

With respect to the plasma measurements, the LS means for vitamin D₃ AUC_{0-120 hr} (not considering endogenous vitamin D₃ serum concentrations) were 296.4 and 337.9 ng·h/mL for the 70 mg alendronate/2800 IU vitamin D₃ combination tablet and 2800 IU vitamin D₃ tablet, respectively (Table 7-6). The AUC_{0-120 hr} GMR (alendronate plus vitamin D₃ combination tablet/vitamin D₃ tablet) was 0.88, with a 90% CI of (0.81, 0.95).

TABLE 7-6
Summary Statistics and GMR With Corresponding 90% Confidence Intervals for
Vitamin D₃ AUC_{0-120 hr} (ng · hr/mL) Not Considering Endogenous
Vitamin D₃ Serum Concentrations Following Single-Dose Administration of 70 mg
Alendronate Plus 2800 IU Vitamin D₃ Combination Tablet or 2800 IU Vitamin
D₃ Tablet Alone

| | | | | | | Between- | | |
|---|---|---|---|---|---|---|---|---|
| | | | | | | Subject | | 90% CI^{\|\|} |
| Treatment | N | LS Mean^{†} | Median | Min | Max | SD^{‡} | GMR^{§} | for GMR |
| Alendronate/ | 28 | 296.4 | 257.5 | 85.0 | 1648.8 | 375.5 | 0.88 | (0.81, |
| Vitamin D₃ | | | | | | | | 0.95) |
| Vitamin D₃ | 28 | 337.9 | 309.6 | 111.9 | 1485.9 | 344.2 | | |
| Alone | | | | | | | | |

Root Mean Squared Error (RMSE) = 0.168 (from the ANOVA model).
^{†}Least-square Means back-transformed from the log scale.
^{‡}SD = Standard Deviation back-transformed from log scale.
^{§}GMR = Geometric Mean Ratio (LS mean of vit D₃ + alendronate/LS mean of vit D₃).
^{||}CI = Confidence Interval.

The LS means for vitamin D₃ Cₘₐₓ, not considering endogenous vitamin D₃ serum concentrations, were 5.9 and 6.6 ng/mL for 70 mg alendronate/2800 IU vitamin D₃ combination tablet and 2800 IU vitamin D₃ tablet, respectively (Table 7-7). The GMR for Cₘₐₓ (alendronate plus vitamin D₃ combination tablet/vitamin D₃ tablet) was 0.89, with a 90% CI of (0.84, 0.95). The 90% CI for AUC_{0-120 hr} and Cₘₐₓ GMR not considering for vitamin D₃ serum concentrations, fell within the pre-specified bioequivalence bounds of (0.80, 1.25).

TABLE 7-7
Summary Statistics and GMR With Corresponding 90% Confidence Intervals for
Vitamin D₃ Cₘₐₓ (ng/mL) Not Considering Endogenous
Vitamin D₃ Serum Concentrations Following Single-Dose Administration of 70 mg
Alendronate Plus 2800 IU Vitamin D₃ Combination Tablet or 2800 IU Vitamin
D₃ Alone Tablet

| | | | | | | Between- | | |
|---|---|---|---|---|---|---|---|---|
| | | | | | | Subject | | 90% CI^{\|\|} |
| Treatment | N | LS Mean^{†} | Median | Min | Max | SD^{‡} | GMR^{§} | for GMR |
| Alendronate/ | 28 | 5.9 | 5.3 | 2.5 | 17.4 | 3.3 | 0.89 | (0.84, |
| Vitamin D₃ | | | | | | | | 0.95) |
| Vit D₃ Alone | 28 | 6.6 | 6.2 | 3.5 | 18.1 | 3.1 | | |

Root Mean Squared Error (RMSE) = 0.138 (from the ANOVA model).
^{†}Least-Square Means back-transformed from the log scale.
^{‡}SD = Standard Deviation back-transformed from log scale.
^{§}GMR = Geometric Mean Ratio (LS mean of vit D₃ + alendronate/LS mean of vit D₃).
^{||}CI = Confidence Interval.

Table 7-8 displays the results of statistical analysis for the vitamin D₃ Tₘₐₓ, obtained from serum profiles not considering endogenous vitamin D₃ serum concentrations. The median Tₘₐₓ for vitamin D₃ with and without alendronate was 12.0 and 9.0 hours, respectively. No significant between-treatment difference was observed (p-value>0.200).

TABLE 7-8
Summary Statistics for Vitamin D₃ Tₘₐₓ (Hours) Obtained from
Serum Profiles Not Considering Endogenous Vitamin
D₃ Serum Concentrations Following Single-Dose Administration of 70 mg
Alendronate Plus 2800 IU Vitamin D₃ Combination Tablet or 2800 IU
Vitamin D₃ Tablet Alone

| | | | | | Between- | |
|---|---|---|---|---|---|---|
| Treatment | N | Median | Min | Max | Subject SD^{†} | p-Value^{‡} |
| Alendronate/ | 28 | 12.0 | 7.0 | 16.0 | 2.6 | 0.978 |
| VitaminD₃ | | | | | | |
| Vitamin | 28 | 9.0 | 7.0 | 16.0 | 2.3 | |
| D₃ Alone | | | | | | |

^{†}SD = Standard Deviation.
^{‡}p-Value was computed using rank analysis.

The LS means for vitamin D₃ AUC_{0-120 hr} were 297.5 and 336.7 ng·h/mL for 70 mg alendronate/2800 IU vitamin D₃ combination tablet and 2800 IU vitamin D₃ tablet, respectively (Table 7-9). The AUC_{0-120 hr} GMR (alendronate plus vitamin D₃ combination tablet/vitamin D₃ tablet) was 0.88, with a 90% CI of (0.82, 0.95).

TABLE 7-9
Summary Statistics and GMR With Corresponding 90% Confidence
Intervals for Vitamin D₃ AUC_{0-120 hr} (ng · h/mL), with Predose Vitamin
D₃ Concentration at Time = 0 as a Covariate, Following Single-Dose
Administration of 70 mgAlendronate plus 2800 IU Vitamin D₃
Combination Tablet or 2800 IU Vitamin
D₃ Alone Tablet

| | | | | | | Between- | | |
|---|---|---|---|---|---|---|---|---|
| | | LS | | | | Subject | | 90% CI^{\|\|} for |
| Treatment | N | Mean^{†} | Median | Min | Max | SD^{‡} | GMR^{§} | GMR |
| Alendronate/ | 28 | 297.5 | 257.5 | 85.0 | 1648.8 | 376.8 | 0.88 | (0.82, 0.95) |
| Vitamin D₃ | | | | | | | | |
| Vitamin D₃ | 28 | 336.7 | 309.6 | 111.9 | 1485.9 | 343.0 | | |
| Alone | | | | | | | | |

Root Mean Squared Error (RMSE) = 0.154 (from the ANOVA model).
^{†}Least-Square Means back-transformed from the log scale.
^{‡}SD = Standard Deviation back-transformed from log scale.
^{§}GMR = Geometric Mean Ratio (LS mean of vit D₃ + alendronate/LS mean of vit D₃).
^{||}CI = Confidence Interval.

The LS means Cₘₐₓ of vitamin D₃, were 5.9 and 6.6 ng/mL for 70 mg alendronate/2800 IU vitamin D₃ combination tablet and 2800-IU vitamin D₃ tablet, respectively, as shown in Table 7-10 below. The Cₘₐₓ GMR (70 mg alendronate/2800 IU vitamin D₃ combination tablet/2800 IU vitamin D₃ tablet) was 0.90, with a 90% CI of (0.85, 0.95). The 90% CI for AUC_{0-120 hr} and Cₘₐₓ GMR with pre-dose vitamin D₃ concentration at time=0 as a covariate, fell within the pre-specified bioequivalence bounds of (0.80, 1.25).

TABLE 7-10
Summary Statistics and GMR With Corresponding 90% Confidence Intervals for Vitamin
D₃ Cₘₐₓ (ng/mL) With Predose Vitamin D₃ Concentration at Time = 0 as a Covariate,
Following Single-Dose Administration of 70 mg Alendronate plus 2800 IU Vitamin D₃
Combination Tablet or 2800 IU Vitamin D₃ Alone Tablet

| | | | | | | Between- | | |
|---|---|---|---|---|---|---|---|---|
| | | LS | | | | Subject | | |
| Treatment | N | Mean^{†} | Median | Min | Max | SD^{‡} | GMR^{§} | 90% CI^{\|\|} for GMR |
| Alendronate/ | 28 | 5.9 | 5.3 | 2.5 | 17.4 | 3.3 | 0.90 | (0.85, 0.95) |
| Vitamin D₃ | | | | | | | | |
| Vitamin D₃ | 28 | 6.6 | 6.2 | 3.5 | 18.1 | 3.1 | | |
| Alone | | | | | | | | |

Root Mean Squared Error (RMSE) = 0.115 (from the ANOVA model).
^{†}Least-Square Means back-transformed from the log scale.
^{‡}SD = Standard Deviation back-transformed from log scale.
^{§}GMR = Geometric Mean Ratio (LS mean of vit D₃ + alendronate/LS mean of vit D₃).
^{||}CI = Confidence Interval.

The results of the data analysis of the AUC_{0-120 hr} of vitamin D₃, measured using model-based vitamin D₃ baseline concentrations, are summarized in Table 7-11. The LS means for vitamin D₃ AUC_{0-120 hr} measured using model-based vitamin D₃ baseline concentrations were 143.1 and 169.1 ng·h/mL for the 70 mg alendronate/2800 IU vitamin D₃ combination tablet and the 2800 IU vitamin D₃ tablet, respectively. The AUC_{0-120 hr} GMR (alendronate plus vitamin D₃ combination tablet/vitamin D₃ tablet) was 0.85, with a 90% CI of (0.76, 0.94). The lower limit of 90% CI fell just below the pre-specified lower bound of 0.80.

TABLE 7-11
Summary Statistics and GMR With Corresponding 90% Confidence Intervals for Vitamin
D₃ AUC_{0-120 hr} (ng · hr/mL), Measured Using Model-Based Vitamin D₃ Baseline
Concentrations, Following Single-Dose Administration of 70 mg Alendronate/2800 IU
Vitamin D₃ Combination Tablet

| | | | | | | Between- | | |
|---|---|---|---|---|---|---|---|---|
| | | LS | | | | Subject | | |
| Treatment | N | Mean^{†} | Median | Min | Max | SD^{‡} | GMR^{§} | 90% CI^{\|\|} for GMR |
| Alendronate/ | 28 | 143.1 | 153.5 | 61.1 | 236.1 | 47.7 | 0.85 | (0.76, 0.94) |
| Vitamin D₃ | | | | | | | | |
| Vit D₃ Alone | 28 | 169.1 | 175.0 | 107.2 | 251.4 | 37.3 | | |

Root Mean Squared Error (RMSE) = 0.224 (from the ANOVA model).
^{†}Least-Square Means back-transformed from the log scale.
^{‡}SD = Standard Deviation back-transformed from log scale.
^{§}GMR = Geometric Mean Ratio (LS mean of vit D₃ + alendronate/LS mean of vit D₃).
^{||}CI = Confidence Interval.

The LS means Cₘₐₓ of vitamin D₃, measured using model-based vitamin D₃ baseline concentrations, were 4.0 and 4.6 ng/mL for the 70 mg alendronate/2800 IU vitamin D₃ combination tablet and 2800 IU vitamin D₃ tablet, respectively, as shown in Table 7-12. The Cₘₐₓ GMR (70 mg alendronate/2800 IU vitamin D₃ combination tablet/2800 IU vitamin D₃ tablet) was 0.88, with a 90% CI of (0.83, 0.93). The 90% CI for Cₘₐₓ GMR measured using model-based vitamin D₃ baseline concentrations fell within the pre-specified bioequivalence bounds of (0.80, 1.25).

TABLE 7-12
Summary Statistics and GMR With Corresponding 90% Confidence Intervals for Vitamin
D₃ Cₘₐₓ (ng/mL) Measured Using Model-Based Vitamin D₃ Baseline Concentrations Following
Single-Dose Administration of 70 mg Alendronate plus 2800 IU Vitamin D₃ Combination
Tablet or 2800 IU Vitamin D₃ Tablet Alone

| | | | | | | Between- | | |
|---|---|---|---|---|---|---|---|---|
| | | LS | | | | Subject | | |
| Treatment | N | Mean^{†} | Median | Min | Max | SD^{‡} | GMR^{§} | 90% CI^{\|\|} for GMR |
| Alendronate/ | 28 | 4.0 | 4.0 | 1.9 | 6.0 | 1.1 | 0.88 | (0.83, 0.93) |
| Vitamin D₃ | | | | | | | | |
| Vitamin D₃ | 28 | 4.6 | 4.6 | 3.0 | 7.2 | 0.9 | | |
| Alone | | | | | | | | |

Root Mean Squared Error (RMSE) = 0.115 (from the ANOVA model).
^{†}Least-Square Means back-transformed from the log scale.
^{‡}SD = Standard Deviation back-transformed from log scale
^{§}GMR = Geometric Mean Ratio (LS mean of vit D₃ + atendronate/LS mean. of vit D₃).
^{||}CI = Confidence Interval.

Table 7-13 displays the results of statistical analysis for vitamin D₃ Tₘₐₓ obtained from profiles measured using model-based baseline vitamin D₃ concentrations. The median Tₘₐₓ for vitamin D₃ with or without alendronate was 12.0 and 9.0 hours, respectively. No significant between-treatment difference was observed (p-value>0.200).

TABLE 7-13
Summary Statistics for Vitamin D₃ Tₘₐₓ (Hours) Obtained From Profiles
Measured Using Model-Based Baseline Vitamin D₃ Concentrations
Following Single-Dose Administration of 70 mg Alendronate Plus 2800
IU Vitamin D₃ Combination Tablet or 2800 IU Vitamin D₃ Tablet Alone

| | | | | | Between- | |
|---|---|---|---|---|---|---|
| Treatment | N | Median | Min | Max | Subject SD^{†} | p-Value^{‡} |
| Alendronate/ | 28 | 12.0 | 7.0 | 16.0 | 2.6 | 0.978 |
| Vitamin D₃ | | | | | | |
| Vitamin D₃ | 28 | 9.0 | 7.0 | 16.0 | 2.3 | |
| Alone | | | | | | |

^{†}SD = Standard Deviation.
^{‡}p-Value was computed using rank analysis.

The results of the data analysis of the t_{1/2} of vitamin D₃, measured using model-based vitamin D₃ background concentrations, are summarized in Table 7-14. The harmonic mean apparent t/, for vitamin D₃ with and without alendronate was 24.0 and 25.5 hours, respectively. No significant between-treatment difference was observed (p-value>0.200).

TABLE 7-14
Summary Statistics for Apparent t_{1/2} (hours) For Vitamin D₃ Measured Using Model-Based
Vitamin D₃ Baseline Concentrations Following Single-Dose Administration of 70 mg
Alendronate/2800 IU Vitamin D₃ Combination Tablet or 2800 IU Vitamin D₃ Alone Tablet

| | | | Jackknife | | | | |
|---|---|---|---|---|---|---|---|
| | | Harmonic | Between-Subject | | | | |
| Treatment | N | Mean | SD^{†} | Min | Median | Max | p-Value^{‡} |
| Vit D₃ + Alendronate | 28 | 23.8 | 11.9 | 11.5 | 24.0 | 100.8 | 0.833 |
| Vit D₃ | 28 | 23.2 | 18.5 | 5.3 | 25.5 | 188.0 | |

^{†}SD = Standard Deviation
^{‡}p-Value was computed using inverse transformation

### Example 8

Degradation Detection Method

A method has been developed for the composite assay of vitamin D₃ in combination alendronate/vitamin D₃ tablets (70 mg alendronate/2800 IU vitamin D₃). Vitamin D₃ is extracted from 15 tablets in about 50 mL of 5% water/95% methanol diluent. The solution is stirred for 10 minutes, sonicated for 30 minutes, and stirred for an additional 3 hours. Samples are centrifuged and 100 μL of the supernatant are injected onto a Phenomenex Phenosphere 80 Å ODS (1) column (150×4.6 mm, 3 μm) for reversed phase HPLC analysis. The method is a 65-minute gradient method with a detection wavelength at 265 nm. Both pre-vitamin D₃ and vitamin D₃ peaks are quantitated and summed to calculate the total amount of vitamin D₃ in the sample. The method was validated for satisfactory specificity, linearity, recovery, precision, reproducibility, solution stability, sensitivity, and robustness.

Exemplary Chromatographic Conditions are Listed Below:

| | |
|---|---|
| Flow Rate: | 1.2 mL/min |
| Column Temperature: | 25° C. |
| Injection Volume: | 100 μL |
| Mobile Phase: | Gradient, A = 0.025% phosphoric acid, |
| | B = 99% Acetonitrile/1% A |
| Run Time: | 65 minutes |
| Column: | Phenosphere 80 Å, ODS (1) column, |
| | 150 × 4.6 mm, 3 μm |
| Sample Tray Temperature | 5° C. |
| Detector Wavelength: | 265 nm |

Gradient Time Table:

| | T (min) | | | | | | |
|---|---|---|---|---|---|---|---|
| | 0 | 16 | 39 | 43 | 57 | 57.01 | 65 |
| % Aqueous | 51.5 | 13 | 0 | 0 | 0 | 51.5 | 51.5 |
| % Mixture | 48.5 | 87 | 90 | 100 | 100 | 48.5 | 48.5 |

Formulation Composition of Combination Tablets (70 mg Alendronate/2800 IU Vitamin D₃)

| Composition | Unit Weight (mg) | Weight % |
|---|---|---|
| Alendronate Sodium | 91.5 | 28.2% |
| Dry Vitamin D₃ 100 granules | 26.7* | 8.2% |
| Avicel PH102 | 131 | 40.3% |
| Lactose, Anhydrous | 62.2 | 19.1% |
| Croscarmellose Sodium | 9.75 | 3.00% |
| Colloidal Silica | 0.81 | 0.25% |
| Intragranular Mg Stearate | 2.28 | 0.70% |
| Extragranular Mg Stearate | 0.81 | 0.25% |
| Tablet Weight: | 325 | 100% |

*26.7 grams of the Dry Vitamin D₃ 100 granules contains 105,000 IU/g of vitamin D₃

The excipient peaks and degradates with their typical retention time and relative retention times (RRT) with respect to vitamin D₃ are presented in Table 8-1. Major degradation pathways of vitamin D₃ are photoisomerization, thermal isomerization, and transesterification, as shown in FIG. 5.

TABLE 8-1
Summary of Peak Identification for Combination Tablets

| Retention Time | | |
|---|---|---|
| (minutes) | RRT | Classification |
| 3.08 | 0.09 | Excipient-related |
| 3.63 | 0.10 | Excipient-related |
| 3.88 | 0.108 | Excipient-related |
| 4.05 | 0.11 | Excipient-related |
| 4.23 | 0.12 | Excipient-related |
| 4.78 | 0.13 | Excipient-related |
| 7.53 | 0.21 | Unknown |
| 11.33 | 0.32 | Excipient-related |
| 17.25 | 0.48 | Unknown |
| 17.47 | 0.49 | Excipient-related |
| 18.05 | 0.50 | Excipient-related |
| 20.64 | 0.57 | Excipient-related |
| 26.70 | 0.74 | Trans-vitamin D₃ |
| 28.15 | 0.78 | Vitamin D₃ isomer |
| 31.33 | 0.87 | Pre-vitamin D₃ |
| 34.42 | 0.96 | Vitamin D₃ isomer |
| 35.94 | 1.00 | Vitamin D₃ |
| 39.17 | 1.09 | Vitamin D₃ Isomer |
| 43.45 | 1.21 | Excipient-related |
| 49.85 | 1.39 | C8-vitamin D₃ ester |
| 50.42 | 1.40 | C8-pre-vitamin D₃ ester |
| 54.52 | 1.52 | C10-vitamin D₃ ester |
| 55.65 | 1.55 | C10-pre-vitamin D₃ ester |

The limit of quantitation (LOQ) was determined by injecting different concentrations of vitamin D₃ solution and selecting the lowest concentration with a signal-to-noise ratio above 10. The LOQ was determined as about 9 ng/mL (injection volume 100 μL), which is 0.04% of the method concentration with an average signal-to-noise ratio of 11.1 for ten replicate determinations.

### Example 9

Once-Weekly Dosing Regimens

Alendronate and vitamin D tablets or other solid dosageformulations containing about 70 mg of alendronate, on an alendronic acid active basis, and about 5,600 IU of vitamin D may be prepared. (See Examples 1, 2, and 3). The tablets or other solid dosage formulations may be orally administered to a subject once-weekly, i.e., preferably about once every seven days (for example, every Sunday), for a period of at least one year. This method of administration is expected to be useful and convenient for treating or preventing osteoporosis while providing vitamin D nutrition. This method is also expected to be useful for improving subject acceptance and compliance, and ensuring that all subjects taking a bisphosphonate receive adequate vitamin D nutrition.

Alternatively, alendronate and vitamin D tablets or other solid dosage formulations containing about 70 mg of alendronate, on an alendronic acid active basis, and about 2,800 IU of vitamin D may be prepared. (See, e.g., Examples 1 and 3). The tablets or other solid dosage formulations may be orally administered to a subject once-weekly, i.e., preferably about once every seven days (for example, every Sunday), for a period of at least one year. This method of administration is expected to be useful and convenient for treating or preventing osteoporosis while providing vitamin D nutrition. This method is also expected to be useful for improving subject acceptance and compliance, and ensuring that all subjects taking a bisphosphonate receive adequate vitamin D nutrition.

Alternatively, alendronate and vitamin D tablets or other solid dosage formulations containing about 35 mg to about 70 mg of alendronate, on an alendronic acid active basis, and 2,800 IU of vitamin D may be prepared. (See, e.g., Example 3). The tablets or other solid dosage formulations may be orally administered to a human subject once-weekly, i.e., preferably about once every seven days (for example, every Sunday), for a period of at least one year. This method of administration is expected to be useful and convenient for treating or preventing osteoporosis while providing vitamin D nutrition. This method is also expected to be useful for improving subject acceptance and compliance, and ensuring that all subjects taking a bisphosphonate receive adequate vitamin D nutrition.

Alendronate and vitamin D tablets or other solid dosage formulations containing about 280 mg of alendronate, on an alendronic acid active basis, and about 5,600 IU of vitamin D may be prepared. (See, e.g., Examples 2 and 3). The tablets or other solid dosageformulations may be orally administered to a subject once-weekly, i.e., preferably about once every seven days (for example, every Sunday), for a period of at least one to six months. This method of administration is expected to be useful and convenient for treating Paget's disease while providing vitamin D nutrition. This method is also expected to be useful for improving subject acceptance and compliance, and ensuring that all subjects taking a bisphosphonate receive adequate vitamin D nutrition.

Alternatively, alendronate and vitamin D tablets or other solid dosage formulations containing about 280 mg of alendronate, on an alendronic acid active basis, and about 2,800 IU of vitamin D may be prepared. (See, e.g., Example 3). The tablets or other solid dosageformulations may be orally administered to a subject once-weekly, i.e., preferably about once every seven days (for example, every Sunday), for a period of at least one to six months. This method of administration is expected to be useful and convenient for treating Paget's disease while providing vitamin D nutrition. This method is also expected to be useful for improving subject acceptance and compliance, and ensuring that all subjects taking a bisphosphonate receive adequate vitamin D nutrition.

Alendronate and vitamin D tablets or other solid dosage formulations containing about 280 mg of alendronate, on an alendronic acid active basis, and 5,600 IU or 2,800 IU of vitamin D may be prepared. (See, e.g., Examples 1, 2, and 3). The tablets or other solid dosage formulations may be orally administered to a subject once-weekly, i.e., preferably about once every seven days (for example, every Sunday). This method of administration is expected to be useful and convenient for treating metastatic bone disease while providing vitamin D nutrition. This method is also expected to be useful for improving subject acceptance and compliance, and ensuring that all subjects taking a bisphosphonate receive adequate vitamin D nutrition.

It will be apparent to those skilled in the art that various modifications can be made to this invention of methods and compositions for inhibiting bone resorption without departing from the scope or spirit of the invention or of the claims. It is also intended that the present invention and appended claims cover modifications, variations and equivalents of the methods and compositions for inhibiting bone resorption of the present invention.

1. A pharmaceutical composition, comprising:
a bisphosphonate, pharmaceutically acceptable salts, derivatives or hydrates of the bisphosphonate, or mixtures thereof; and a vitamin D compound.

2. The pharmaceutical composition of claim 1, wherein the bisphosphonate is of the formula: 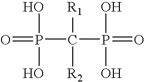
wherein R₁ is independently selected from H, OH, and C₁, R₂ is independently selected from CH₃, Cl, CH₂CH₂NH₂, (CH₂)₃NH₂, CH₂-3-pyridyl, CH₂—S-phenyl-Cl, CH₂CH₂N(CH₃)(pentyl), CH₂-imidazole, CH₂-2-imidazo-pyridinyl, N-(cycloheptyl), CH₂CH(CH₃)₂, (CH₂)₅NH₂, and CH₂-1-pyrrolidinyl, and combinations thereof.

3. The pharmaceutical composition of claim 1, wherein the bisphosphonate comprises alendronate or a pharmaceutically acceptable salt thereof.

4. The pharmaceutical composition of claim 3, wherein the pharmaceutically acceptable salt of alendronate is selected from alendronate monosodium, alendronate monosodium monohydrate, and alendronate monosodium trihydrate.

5. The pharmaceutical composition of claim 1, wherein the pharmaceutical composition comprises from about 100 IU to about 36,000 IU of the vitamin D compound.

6. The pharmaceutical composition of claim 5, wherein the vitamin D compound is cholecalciferol.

7. The pharmaceutical composition of claim 1, wherein the pharmaceutical composition comprises from about 0.5 mg to about 1120 mg of the bisphosphonate, or pharmaceutically acceptable salts, derivatives or hydrates of the bisphosphonate, or mixtures thereof.

8. The pharmaceutical composition of claim 1, wherein the pharmaceutical composition comprises about 2,800 IU of cholecalciferol and about 70 mg of alendronate or pharmaceutically acceptable salts, derivatives or hydrates of alendronate, or mixtures thereof.

9. The pharmaceutical composition of claim 1, wherein the pharmaceutical composition comprises about 5,600 IU of cholecalciferol and about 70 mg of alendronate or pharmaceutically acceptable salts, derivatives or hydrates of alendronate, or mixtures thereof.

10. The pharmaceutical composition of claim 1, further comprising one or more excipients selected from the group consisting of fillers, diluents, binders, lubricants, glidants, and disintegrants.

11. The pharmaceutical composition of claim 1, further comprising one or more excipients selected from the group consisting of lactose anhydrous, microcrystalline cellulose, colloidal silicon dioxide, croscarmellose sodium, and magnesium stearate.

12. The pharmaceutical composition of claim 11, wherein the pharmaceutical composition comprises about 0.5% to about 90% alendronate sodium by weight, about 1% to about 70% cholecalciferol granule by weight (equivalent to about 0.0005% to about 20% cholecalciferol by weight), about 10% to about 80% lactose anhydrous by weight, about 5% to about 50% microcrystalline cellulose by weight, about 0.1% to about 5% colloidal silicon dioxide by weight, about 0.5% to about 10% croscarmellose sodium by weight, and about 0.5% to about 5% magnesium stearate by weight

13. The pharmaceutical composition of claim 6, wherein the cholecalciferol comprises pharmaceutical grade cholecalciferol.

14. The pharmaceutical composition of claim 13, wherein the pharmaceutical composition is suitable for administration at intervals of once-weekly, bi-weekly, monthly, twice-monthly, and bi-monthly.

15. A method for preventing, inhibiting, reducing or treating metabolic bone disease in a mammal, comprising administering to the mammal a pharmaceutical composition comprising:
a bisphosphonate, pharmaceutically acceptable salts, derivatives or hydrates of the bisphosphonate, or mixtures thereof; and a vitamin D compound.

16. The method of claim 15, wherein the bisphosphonate comprises alendronate or a pharmaceutically acceptable salt thereof.

17. The method of claim 15, wherein the vitamin D compound is cholecalciferol.

18. The method of claim 17, wherein the vitamin D compound comprises from about 100 IU to about 36,000 IU cholecalciferol, and wherein the bisphosphonate compound comprises from about 0.5 mg to about 1120 mg of alendronate, or pharmaceutically acceptable salts, derivatives or hydrates of the alendronate, or mixtures thereof.

19. The method of claim 15, wherein the bisphosphonate comprises alendronate monosodium trihydrate and the pharmaceutical composition comprises from about 2.5 mg to about 560 mg of alendronate monosodium trihydrate.

20. The method of claim 18, wherein the pharmaceutical composition comprises about 2800 IU cholecalciferol, and about 70 mg alendronate or pharmaceutically acceptable salts, derivatives or hydrates of alendronate, or mixtures thereof.

21. The method of claim 15, wherein the metabolic bone disease is selected from osteoporosis, post-menopausal osteoporosis, steroid-induced osteoporosis, male osteoporosis, other disease-induced osteoporosis, idiopathic osteoporosis, and glucocorticoid-induced osteoporosis.

22. The method of claim 15 comprising administering to a mammal having vitamin D deficiency or insufficiency a pharmaceutical composition comprising:
a bisphosphonate, pharmaceutically acceptable salts, derivatives or hydrates of the bisphosphonate, or mixtures thereof, and cholecalciferol.

23. A method for preventing, inhibiting, reducing or treating an arthritic condition in a mammal, comprising administering to the mammal a pharmaceutical composition of claim 1 comprising:
a bisphosphonate, pharmaceutically acceptable salts, derivatives or hydrates of the bisphosphonate, or mixtures thereof; and a vitamin D compound.

24. A method for preventing, inhibiting, reducing or treating bone resorption in a mammal comprising orally administering to the mammal the pharmaceutical composition of claim 1, wherein the pharmaceutical composition is administered as a unit dosage according to a continuous dosing schedule having a dosing interval of once weekly.

25. A method for preventing, inhibiting, reducing or treating osteoporosis in a mammal comprising orally administering to the mammal the pharmaceutical composition of claim 1, wherein the pharmaceutical composition is administered as a unit dosage according to a continuous dosing schedule having a dosing interval of once weekly.

26. A method for reducing the risk of bone fractures in a mammal comprising orally administering to the mammal the pharmaceutical composition of claim 1, wherein the pharmaceutical composition is administered as a unit dosage according to a continuous dosing schedule having a dosing interval of once weekly.

27. A method for preparing an alendronate-cholecalciferol composition, comprising:
preparing a powder blend comprising alendronate;
compacting the powder blend to form an alendronate mixture;
milling and blending the alendronate mixture with cholecalciferol granules to form a final blend; and
lubricating and compressing the final blend.

28. A method for preparing an alendronate-cholecalciferol solid dosage form comprising:
blending alendronate, colloidal silicon dioxide, lactose anhydrous, microcrystalline cellulose, and croscarmellose sodium to form a pre-blend;
blending the pre-blend and magnesium stearate to form a first lubricated mixture;
roller compacting the first lubricated mixture to form compacted ribbons;
milling the compacted ribbons to form a lubricated blend;
blending the lubricated blend with cholecalciferol granules to form a second lubricated mixture; and
compressing the second lubricated mixture into the solid dosage form.

29. A pharmaceutical composition prepared by the method of claim 28.

30. The pharmaceutical composition of claim 1 prepared by a method comprising:
blending ingredients comprising about 0.5% to about 90% by weight alendronate sodium, about 0.1% to about 5% by weight colloidal silicon dioxide, about 10% to about 80% by weight lactose anhydrous, about 5% to about 50% by weight microcrystalline cellulose, about 0.5% to about 10% by weight croscarmellose sodium, and about 0.5% to about 5% by weight magnesium stearate to form a first mixture;
roller compacting the first mixture to form compacted ribbons;
milling the compacted ribbons to form a lubricated blend;
blending the lubricated blend with about 1% to about 70% cholecalciferol granule by weight (equivalent to about 0.0005% to about 20% cholecalciferol by weight) to form a second mixture; and
compressing the second mixture into a solid dosage form.

31. The pharmaceutical composition of claim 6, wherein the composition is formulated to comprise less than about 1% by weight of each isomer of cholecalciferol after storage for 24 months at about <30° C. and about <30% relative humidity.

32. The pharmaceutical composition of claim 6, wherein the composition is formulated to comprise less than about 5% by weight of degradants of cholecalciferol after storage for 24 months at about <30° C. and about <30% relative humidity.

33. A pharmaceutical composition comprising:
a bisphosphonate, pharmaceutically acceptable salts, derivatives or hydrates of the bisphosphonate, or mixtures thereof;
cholecalciferol;
wherein a therapeutic effect of the cholecalciferol is substantially similar to a therapeutic effect of about 400 IU cholecalciferol per day when administered over a week; and wherein the pharmaceutical composition is suitable for once-weekly dosing.

34. A method of measuring cholecalciferol in a pharmaceutical composition of claim 1, comprising:
extracting the cholecalciferol from the pharmaceutical composition into a first solution to form a second solution;
separating a sample containing cholecalciferol from the second solution; and
detecting an amount of cholecalciferol in the sample; wherein the detecting is carried out using reverse-phase high performance liquid chromatography (HPLC) separation.

35. The method of claim 34, wherein the detecting is carried out to detect about 2800 IU to about 5600 IU cholecalciferol per pharmaceutical composition.

36. The method of claim 34, wherein the detecting has a limit of quantitation (LOQ) of cholecalciferol of less than about 9 ng/mL cholecalciferol.

37. The method of claim 34, wherein the detecting is carried out using a reverse-phase HPLC column with no endcapping or partial endcapping.

38. The method of claim 34, wherein the detecting is carried out using a reverse-phase HPLC column with carbon loading of less than about 10% carbon.

39. A method of maintaining within the body of a mammal pharmaceutically effective amounts of cholecalciferol comprising administering once-weekly a pharmaceutical composition of claim 1 comprising:
about 70 mg of a bisphosphonate, pharmaceutically acceptable salts, derivatives or hydrates of the bisphosphonate, or mixtures thereof; and
about 2800 IU of cholecalciferol.

40. The pharmaceutical composition of claim 1, wherein the bisphosphonate is alendronate sodium and a plot of plasma concentration from administration to a mammal of the alendronate sodium of the composition is substantially similar to a plot of plasma concentration from administration to the mammal of 70 mg alendronate sodium in the absence of cholecalciferol.

41. The pharmaceutical composition of claim 1, wherein the bisphosphonate is alendronate sodium and a plot of plasma concentration from administration to a mammal of the cholecalciferol of the composition is substantially similar to a plot of plasma concentration from administration to the mammal of 2800 IU cholecalciferol in the absence of alendronate.

42. The pharmaceutical composition of claim 1, wherein a plot of serum concentration of a mammal over 120 hours after administration of the composition yields at least one of the following:
a least-squares (LS) mean AUC_{(0-120 hr)} of cholecalciferol of about 296.4 ng.h/mL, wherein the pharmacokinetic parameters have been measured without taking into account baseline cholecalciferol serum concentrations;
a least-squares (LS) mean AUC_{(0-120 hr)} of about 297.5 ng.h/mL, wherein the pharmacokinetic parameters have been measured by taking into account baseline cholecalciferol serum concentrations using a predose 0 hr serum cholecalciferol concentration as a covariate; and
a least-squares (LS) mean AUC_{(0-120 hr)} of about 143.1 ng.h/mL, wherein the pharmacokinetic parameters have been measured by taking into account baseline cholecalciferol serum concentrations using a subtraction of estimated baseline cholecalciferol over the 120 hour period.

43. The pharmaceutical composition of claim 1, wherein a plot of plasma concentration a mammal over 120 hours after administration of the composition yields at least one of the following:
a least-squares (LS) mean for steady state maximum plasma concentration (Cₘₐₓ) of over 120 hours of about 5.9 ng/mL, wherein the pharmacokinetic parameters have been measured without taking into account baseline cholecalciferol serum concentrations;
a least-squares (LS) mean for steady state maximum plasma concentration (Cₘₐₓ) of over 120 hours of about 5.9 ng/mL, wherein the pharmacokinetic parameters have been measured by taking into account baseline cholecalciferol serum concentrations using a predose 0 hr serum cholecalciferol concentration as a covariate; and
a least-squares (LS) mean for steady state maximum plasma concentration (Cₘₐₓ) of about 4.0 ng/mL, wherein the pharmacokinetic parameters have been measured by taking into account baseline cholecalciferol serum concentrations using a subtraction of estimated baseline cholecalciferol over the 120 hour period.

44. The pharmaceutical composition of claim 1, wherein a plot of the plasma concentration of cholecalciferol of a mammal over 120 hours after administration of the composition yields:
a steady state maximum plasma concentration (Cₘₐₓ) of cholecalciferol at an arithmetic mean time of occurrence of Cₘₐₓ (Tₘₐₓ) of about 12 hours, and wherein the pharmacokinetic parameters have been measured without taking into account baseline cholecalciferol serum concentrations.

45. The pharmaceutical composition of claim 1, wherein the plasma concentration median apparent half-life (t_{1/2}) of the cholecalciferol of the composition in mammals is about 23.8 hours, and wherein the pharmacokinetic parameters have been measured by taking into account baseline cholecalciferol serum concentrations using a subtraction of estimated baseline cholecalciferol procedure.

46. A method of measuring cholecalciferol in mammal serum, comprising:
administering to a mammal a composition of claim 1 comprising alendronate and cholecalciferol;
obtaining from the mammal a plasma sample;
extracting the cholecalciferol from the plasma sample to form a first solution;
reacting the cholecalciferol in the first solution with a dienophile to form one or more diels-alder addition products of cholecalciferol;
separating the diels-alder addition products of cholecalciferol using high performance liquid chromatography (HPLC) separation; and
detecting an amount of cholecalciferol in the sample using mass spectroscopy.

47. The method of claim 46, further comprising adding a deuterated internal standard cholecalciferol to each mammal plasma sample, and extracting, reacting, separating, and detecting the deuterated internal standard cholecalciferol along with the sample cholecalciferol.

48. The method of claim 46, wherein the detecting has a limit of quantitation (LOQ) of cholecalciferol of less than about 0.5 ng/mL cholecalciferol when 1 mL of plasma is measured.
